(19) United States
(12) Patent Application Publication
(10) Pub. No.: US 2022/0152427 A1
(43) Pub. Date: May 19, 2022

(54) System and Methods Using Ultrasound for Treatment

(71) Applicant: AxioSonic, LLC, Effingham, IL (US)

(72) Inventors: Peter M. Bonutti, Manalapan, FL (US); Justin E. Beyers, Effingham, IL (US); Tonya M. Bierman, Dieterich, IL (US)

(21) Appl. No.: 17/589,408

(22) Filed: Jan. 31, 2022

Related U.S. Application Data
(63) Continuation of application No. 15/011,156, filed on Jan. 29, 2016.
Application No. 15/011,156 is a continuation of application No. PCT/US2015/010843, filed on Jan. 9, 2015.
(60) Provisional application No. 62/063,171, filed on Oct. 13, 2014.
(60) Provisional application No. 61/925,395, filed on Jan. 9, 2014.

(51) Int. Cl. A61N 7/00

(52) U.S. Cl. CPC A61N 7/00; A61B 2017/306

(57) ABSTRACT

A device for treating infection within a subject includes an ultrasound transducer for applying ultrasound to a treatment site of the subject. In some embodiments, the device transmits ultrasound in the range of greater than about 20 kHz to about 5 MHz to site of infection. For example, the device can transmit ultrasound from a location outside the body to a location inside the body, such as a sinus. The ultrasound may be suitable for removing biofilm or decreasing the viscosity of mucus. Ultrasound from the device can also have other therapeutic and diagnostic uses.

### CROSS-REFERENCE TO RELATED APPLICATIONS

The present application is a continuation of International Patent Application No. PCT/US15/10843, which was filed on Jan. 9, 2015, and entitled “SYSTEMS AND METHODS USING ULTRASOUND FOR TREATMENT,” and is hereby incorporated by reference. International Patent Application No. PCT/US2015/010843 claims priority to U.S. Provisional Patent Application No. 61/925,395, filed Jan. 9, 2014, and entitled “SYSTEMS AND METHODS FOR TRANSMITTING HIGH FREQUENCY ULTRASOUND,” and U.S. Provisional Patent Application No. 62/063,171, filed Oct. 13, 2014 and entitled “SYSTEMS AND METHODS FOR TREATING INFECTION USING ULTRASOUND,” each of which is hereby incorporated by reference.

### BACKGROUND

The field of the disclosure relates generally to systems and methods for using ultrasound for treatment in the healthcare field.

Generally, it has been shown that some infections are resistant to conventional treatments, such as antibiotics alone. For example, biofilm and Methicillin-resistant Staphylococcus aureus are resistant to antibiotic treatment alone. It is also known that some treatments in the healthcare field need improvements.

### SUMMARY

In one aspect, a device for treating infection within a subject comprises an ultrasound transducer for applying ultrasound to a treatment site of the subject.

The features, functions, and advantages that have been discussed can be achieved independently in various embodiments or may be combined in yet other embodiments, further details of which can be seen with reference to the following description and drawings.

### BRIEF DESCRIPTION OF THE DRAWINGS

FIG. 1 is a perspective view of an exemplary high frequency ultrasound (HFUS) device for affecting bacteria, biofilm, and/or infection within the body.

FIG. 2 is a block diagram of circuitry positioned in the device housing shown in FIG. 1.

FIG. 3 is a cross section of the treatment applicator shown in FIG. 1.

FIG. 4 is a perspective view of an alternative HFUS device having the components of the device shown in FIG. 1.

FIG. 5 is schematic diagram of potential treatment regions of a patient that may be used with the device shown in FIG. 1.

FIG. 6 is a graphical representation of the results of Test 1 using the device shown in FIG. 1.

FIG. 7 displays microscope images of the results of Test 2 using the device shown in FIG. 1.

FIG. 8 displays microscope images of the results of Test 3 using the device shown in FIG. 1.

FIG. 9 is an exemplary flowchart of a method for use with the device shown in FIG. 1.

FIG. 10 is an exemplary flowchart of a method for use with the device shown in FIG. 1.

FIG. 11 is an exemplary drive waveform that may be used with the applicator shown in FIG. 1.

FIG. 12 is an exemplary drive section that may be used with the applicator shown in FIG. 1.

FIG. 13 is an alternative drive section that may be used with the applicator shown in FIG. 1.

FIG. 14 is an alternative drive section that may be used with the applicator shown in FIG. 1.

FIG. 15 is an alternative drive section that may be used with the applicator shown in FIG. 1.

FIG. 16 is a perspective view of an alternative HFUS device having the components of the device shown in FIG. 1.

FIG. 17 is a perspective view of the HFUS device of FIG. 1 with a display showing a graphical representation of a human body.

FIG. 18 is a perspective view of the HFUS device of FIG. 1 with a display showing a graphical representation of a human knee.

FIG. 19 is a schematic showing the HFUS device of FIG. 1 treating a human knee.

FIG. 20 is a schematic showing the HFUS device of FIG. 1 treating a stent in a human body.

FIG. 21 is a schematic showing the HFUS device of FIG. 1 treating a screw in a human body.

FIG. 22 is a schematic showing the HFUS device of FIG. 1 treating mesh in a human body.

FIG. 23 is a cross section of another embodiment of a treatment applicator similar to the treatment applicator of FIG. 1.

FIG. 24 is a perspective of another embodiment of a high frequency ultrasound (HFUS) device for affecting bacteria, biofilm, and/or infection within the body, similar to the embodiment illustrated in FIG. 1.

FIG. 25 is a front elevational view of FIG. 24.

Corresponding parts are given corresponding reference numbers throughout the drawings.

### DETAILED DESCRIPTION

In one embodiment, the systems and methods described herein enable treatment of infection of a living subject (i.e., a human or other animal) using high frequency ultrasound (HFUS). As used herein, the term “infection” refers to an invasion of the living subject by an infectious agent, regardless of whether the infectious agent causes a disease. Non-limiting examples of infectious agents causing infection include bacteria, viruses, fungi, parasites, and prions. The infectious agent(s) causing the infection may exist in the living subject in a planktonic state or as biofilm. As used herein, infectious agents causing the infection are in a planktonic state (i.e., a planktonic infection) if the infectious agents are free-floating within the subject, and the infectious agents are in a biofilm (i.e., a biofilm infection) if the infectious agents are microorganisms adhered to each other on a surface within the subject and are enclosed by a self-produced matrix of a secreted extracellular polymeric substance. The biofilm extracellular polymeric substance excreted by the biofilm infection may comprise polysaccharides (e.g., exopolysaccharides), proteins, DNA, lipids and humic substances. Examples of infectious agents forming biofilms described herein include, but are not limited to, bacteria, archaea, protozoa, fungi, and algae.

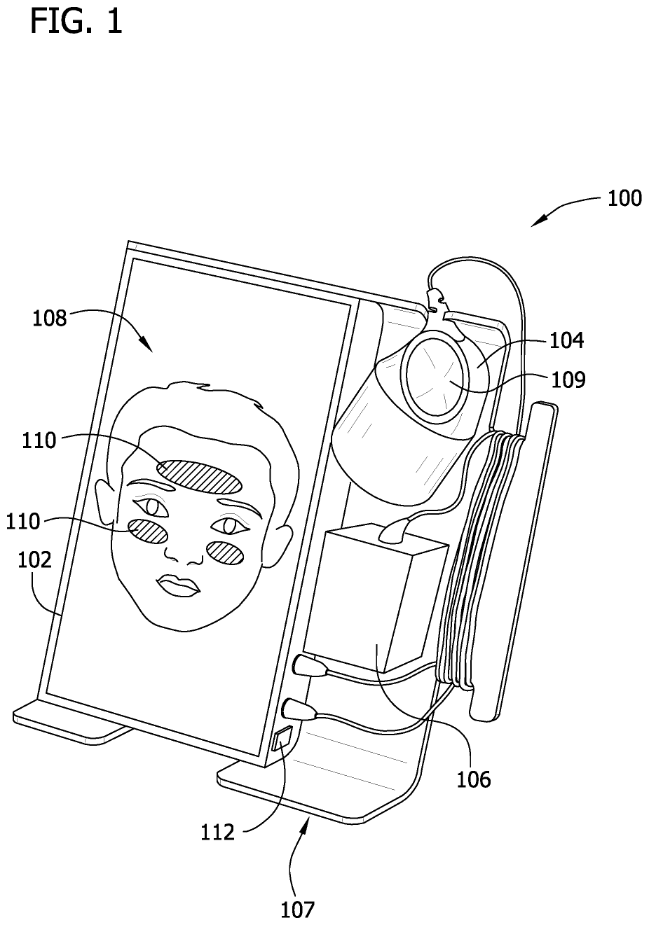

FIG. 1 is a perspective view of an exemplary high frequency ultrasound (HFUS) device, generally indicated at 100, for treating infection of a living subject. In general, the device is configured to deliver ultrasonic energy (e.g., high frequency ultrasonic energy) to a site of infection of the living subject to treat (i.e., combat, ameliorate, inhibit, and/or prevent) the infection. Device 100 comprises a device housing 102, a treatment applicator 104 including an ultrasonic transducer 310, and a control circuit 200 contained within the housing for controlling the output of the ultrasonic transducer. In one embodiment, device 100 is powered by an AC power adapter 106 (e.g., an external or internal AC power adapter) configured to receive AC power from a power source (e.g., mains power) and convert the AC power to DC power used by device 100. In the illustrated embodiment, the HFUS device 100 also includes a DC power source within the housing 102. As a non-limiting example, DC power source may be a battery, including but not limited to, a rechargeable lithium-ion battery (e.g., battery and charger circuit 202). The HFUS device 100 may be powered in other ways without departing from the scope of the present invention.

In one embodiment, applicator 104 and/or housing 102 are configured to be hand-held and portable such that a user can utilize applicator 104 and/or housing 102 with one hand. In some embodiments, applicator 104 and/or housing 102 are configured to have an ergonomic design when held by a user. For example, in the illustrated embodiment applicator 104 includes a recess 109 that contours to one or two fingers that aid in stabilization of applicator 104. Recess 109 also enables a user to hold applicator 104 with a pinch grip for ease of use. Housing 102 and applicator 104 are storable on a base 107 (e.g., a stand). Housing 102 and/or applicator 104 may be removably coupled to base 107, such as by magnets (not shown)

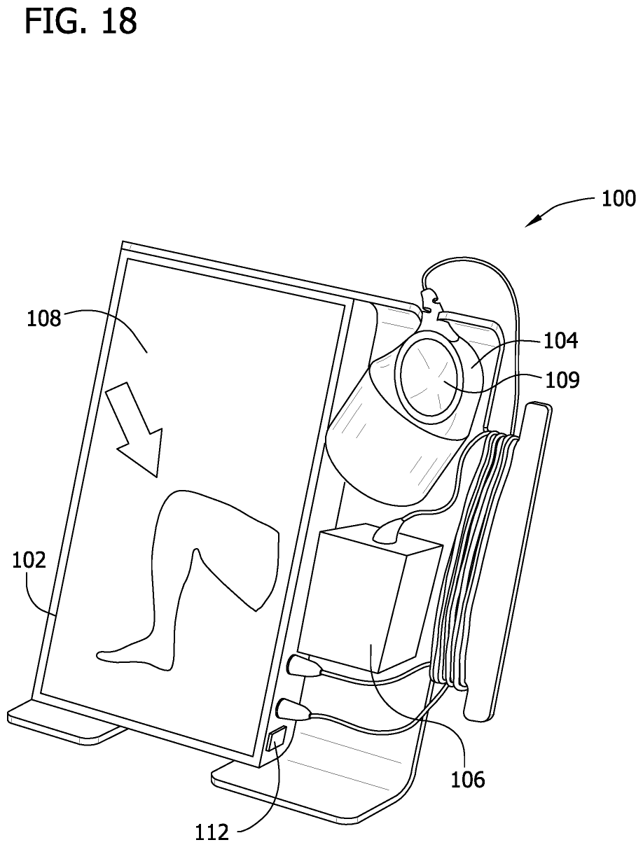

A user interface 108 is provided on housing 102 to allow communication between the user and device 100, in particular between the user and the control circuit 200. User interface 108 has a presentation function configured to present information, such as treatment information and/or execution events, to a user. For example, user interface 108 may include a display device, as illustrated, for presenting information to a user. The display device may include a cathode ray tube (CRT), a liquid crystal display (LCD), LED, an organic LED (OLED) display, a vacuum fluorescent display (VFD), and/or an “electronic ink” display. In some embodiments, user interface 108 may include one or more display devices. In the illustrated embodiment, user interface 108 displays the intended application area and/or configuration of device 100 for treating infection of a user. For example, as illustrated in FIG. 1, user interface 108 comprises a display generating a graphical representation of a human face to which treatment of infection using device 100 is to be applied. In other examples, device 100 may be configured to generate a graphic representation of another portion(s) of a human body or the entire human body on the display. For example, as shown in FIG. 18 a graphical representation of a knee or other joint of the body may be generated on the display to indicate the desired treatment site. Data may be stored in a remote database, such as cloud storage.

In the exemplary embodiment, user interface 108 also has an input function to allow a user to communicate with device 100, in particular control circuit 200. As an example, to allow a user to communicate with device 100, user interface 108 may include keys, a pointing device, a mouse, a stylus, a membrane switch, a touch sensitive panel (e.g., a touch pad or a touch screen), a gyroscope, an accelerometer, a position detector, and/or an audio user input interface. In the illustrated embodiment, user interface 108 comprises a touch screen having both presentation and input functions. In one example, user interface 108 may be configured to receive an input from the user as to a desired treatment area and/or treatment protocol. In the illustrated embodiment user interface 108 (i.e., touch screen) may be configured to allow the user to select a body portion for treatment and/or a specific area of a body portion for treatment. For example, as illustrated user interface 108 allows a user to select a sinus area for treatment by touching the desired sinus area on the display. This selection is communicated to control circuit 200, as explained in more detail below.

In the exemplary embodiment, a communication interface 112 coupled to control circuit 200 is provided on housing 102. Communication interface 112 communicates with control circuit 200 to allow transfer of treatment and/or session information stored by device 100. To communicate with control circuit 200, communication interface 112 may include, for example, a wired network adapter, a wireless network adapter, and/or a mobile telecommunications adapter. In some embodiments, communication interface 112 is a direct link interface for linking two computing devices, the direct link interface including, but not being limited to, a serial port, a firewire port, a USB port, and an Ethernet port. In one embodiment, communication interface 108 includes a Bluetooth adapter capable of communication with a Bluetooth receiver positioned in a separate computing device (e.g., tablet, pc, smartphone, and smartwatch). In some embodiments, communication interface 112 receives information such as executable instructions and/or other data that can be stored and/or executed by control circuit 200.

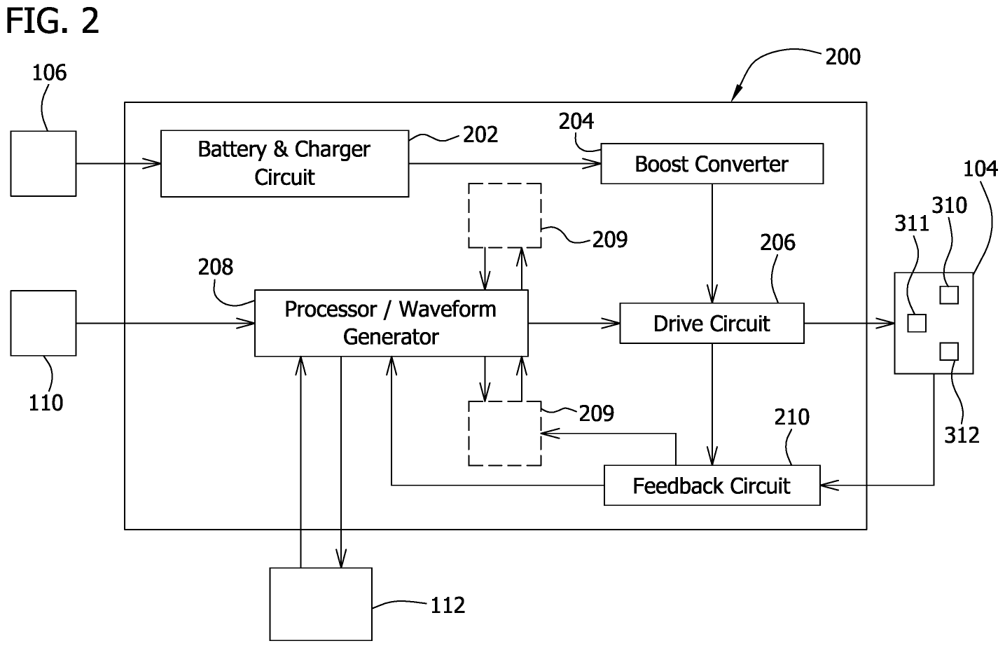

FIG. 2 is a block diagram of device 100 illustrated in FIG. 1. As shown in FIG. 2, power source 106 is electrically connected to a battery and charger circuit 202. Electrical power (e.g., DC current) is delivered from power source 106 (e.g., AC adapter) to battery and charger circuit 202. Battery and charger circuit 202 is electrically connected to boost converter 204. Battery and charger circuit 202 transmits electrical power (e.g., DC power) to boost converter 204. Boost converter 204 is electrically connected to drive circuit 206. Boost converter 204 outputs DC power to drive circuit 206 having a DC voltage that is greater than the DC voltage of the power received from battery and charger circuit 202.

A processor 208 of control circuit 200 is electrically connected to user interface 110, communication interface 112, and drive circuit 206. In the illustrated embodiment, processor 208 is configured to execute instructions provided on non-transitory computer readable medium, such as memory device 209. Instructions provided on memory device 209 include instructions for operating device 100 to treat infection of a subject using applicator 104, as explained below. Processor 208 communicates with user interface 110 to receive commands from a user and output information to the user, as described below. In the illustrated embodiment, processor 208 operates as a waveform generator, whereby an electrical signal is delivered to drive circuit 206 in accordance with the desired frequency output of ultrasound transducer 310. In particular, drive circuit 206 is configured to receive DC power from boost converter 204 and a waveform electrical signal from processor 208. The drive circuit 206 delivers an AC drive signal to transducer 310 of applicator 104 based on the waveform electrical signal and the DC power received from the boost converter 204. Transducer 310 outputs the desired HFUS energy (i.e., having a desired frequency and intensity) in accordance with the received drive signal. The outputted HFUS energy is suitable for treating infection of the subject. The output of transducer 104 may be monitored by a feedback circuit 210 in communication with processor 208.

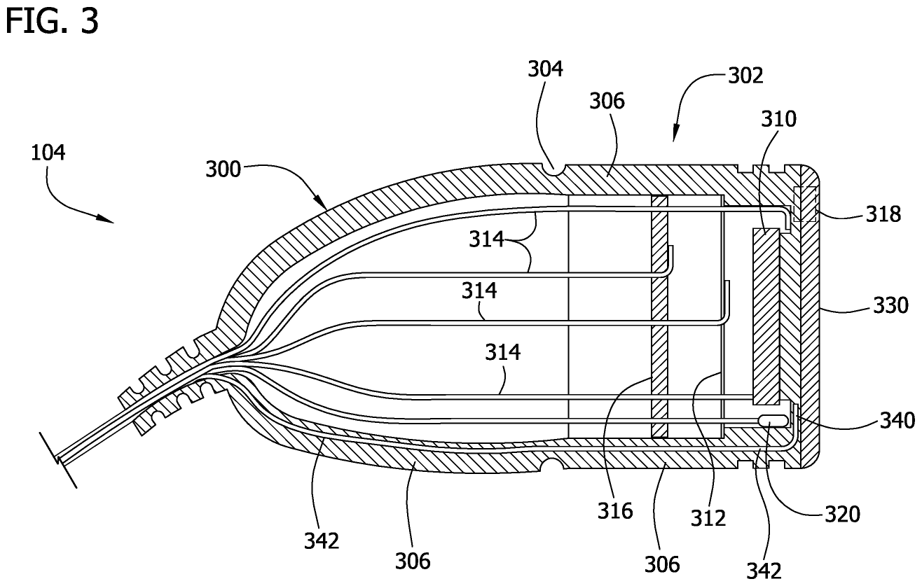

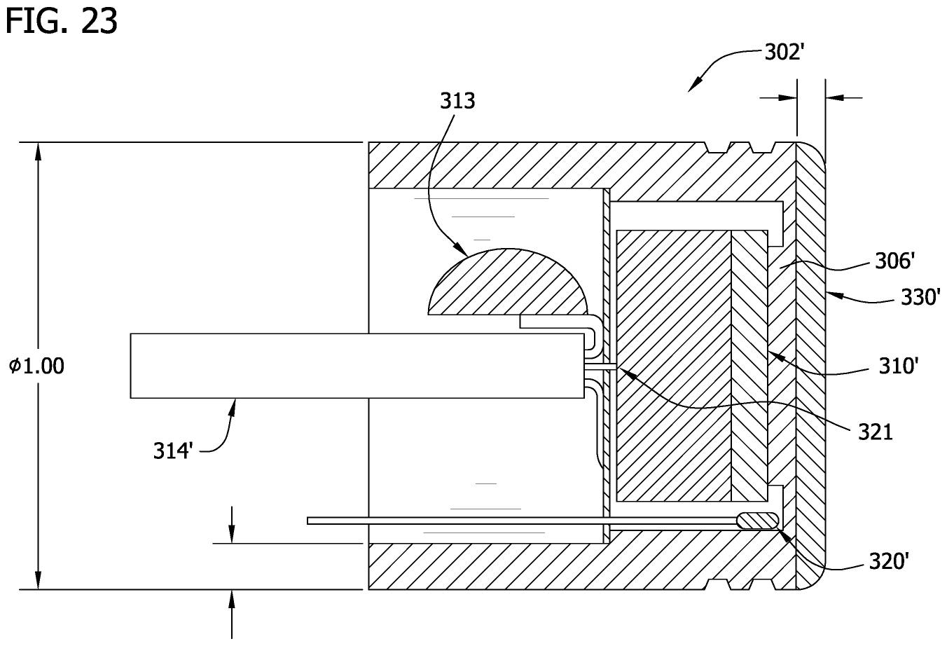

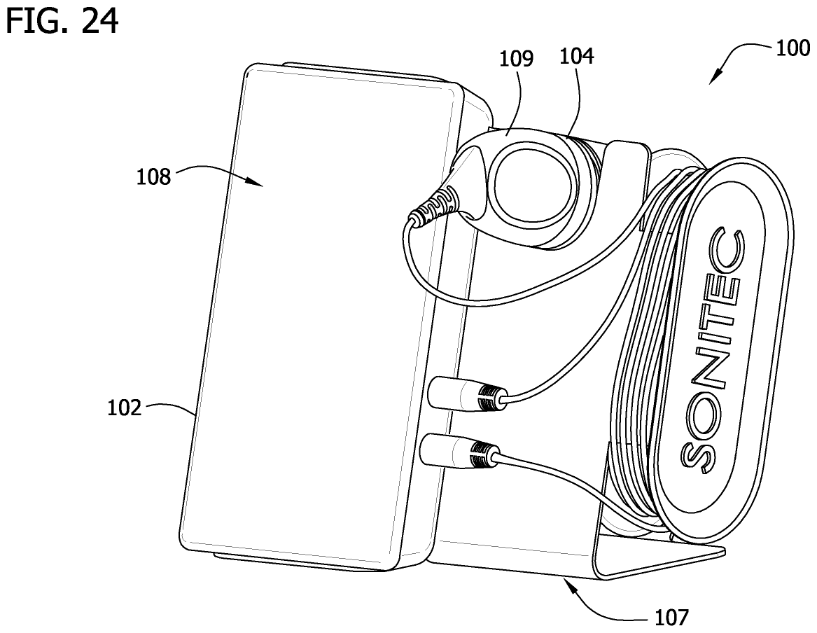

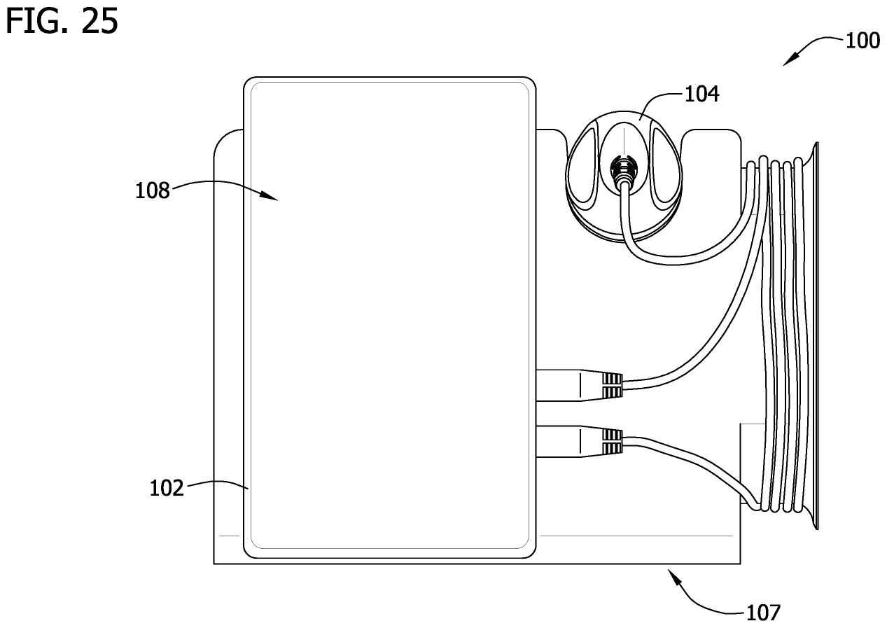

FIG. 3 is a side cut-away view of applicator 104 shown in FIG. 1. In the exemplary embodiment, applicator 104 includes shell 306 having a body portion, generally indicated at 300, and an applicator head portion, generally indicated at 302, configured to provide the HFUS energy to a treatment site in the body. A similar embodiment of a head portion 302′ is shown in FIG. 23, with differences between the embodiments discussed below. In some embodiments, a retention groove 304 is formed on shell 306. Retention groove 304 is configured to enable applicator 104 be held by a system retention apparatus, such as a clip or stand provided on base 107. For example, as shown in FIGS. 24 and 25, in another embodiment the base 107 incudes a U-shaped cutout 305 in which the applicator 104 is retained. A bottom edge defining the U-shaped cutout 305 is received in the retention groove 304 of the applicator 104 to hold the applicator in the U-shaped cutout. In one embodiment, shell 306 of applicator 104 is fabricated from a polymer, including but not limited to, acrylonitrile butadiene styrene, polyether ether ketone, Polyoxymethylene. Alternatively, shell 306 can be fabricated from any material that facilitates transmitting energy (e.g. ultrasonic vibrations) from applicator 104 to a treatment site of the subject, including but not limited to, titanium, aluminum, and stainless steel. In another embodiment, body portion 300 of outer shell 306 may be fabricated from a polymer and head portion 302 may be fabricated from a metallic substance (e.g. titanium).

As described above, transducer 310 is configured to convert drive signal into ultrasonic vibratory energy that will be utilized to treat infection at a treatment site of the subject. In the exemplary embodiment, transducer 310 is configured to output ultrasound energy having a frequency selected to be above 20 kHz, such as from about 1 MHz to about 5 MHz, and an intensity from about 0.20 W/cm² to about 3 W/cm², such as from about 0.5 W/cm² to about 1 W/cm², in accordance with the drive signal received from drive circuit 206. Transducer 310 may comprise a piezoelectric crystal having a square shape or any other suitable shape, including but not limited to, round, circular, oval, and rectangular. In at least some embodiments, applicator 104 includes a plurality or array of transducers 310 for transmitting ultrasonic vibratory energy. In such embodiments, transducers 310 are arranged to focus ultrasound at a treatment site, with two or more transducers 310 outputting different frequencies such that the intersection of the ultrasound beams will create a different frequency at the desired treatment site. Alternatively, discrete transducers 310 are configured to provide different beams that are configured to affect particular portions (e.g. proximal, distal, etc.) of a treatment site. In some embodiments, discrete transducers 310 are configured to simultaneously provide multiple beams that are configured to treat different types of infections (e.g. MRSA infection, bacterial biofilm infection, and fungal biofilm infection).

In some embodiments, the frequency of the output of transducer 310 is 1 MHz. An output of ultrasonic vibratory energy of 1 MHz has a beneficial effect on pain and swelling. Additionally, it is believed the output of ultrasonic vibratory energy at a frequency of 1 MHz has an effect on infectious agents and/or biofilms by turning infectious agents, such as bacteria, and/or biofilm into planktonic state, causing delamination of biofilm, creating a physical disruption, and/or breakdown of polysaccharides present in biofilm matrix. Further, with respect to treating infection of a sinus cavity, the application of the high frequency ultrasound can have an effect on the viscosity of mucus in the sinus cavity which can enhance drainage.

In the exemplary embodiment (FIG. 1), applicator 104 includes a vibratory device 312, such as but not limited to a piezoelectric transducer or an eccentric vibrator motor, that provides tactile vibrations to the user. Such an embodiment enables a user to feel that applicator 104 is functioning and/or in a treatment mode. Vibratory device 312 may be configured to operate, and thereby vibrate, when the ultrasound transducer 310 is outputting the ultrasound signal during treatment. In one embodiment, vibratory device 312 may be powered by the drive signal from drive circuit 206, while the drive signal simultaneously powers ultrasound transducer 310. In the exemplary embodiment, ultrasonic transducer 310 and vibratory transducer 312 are configured to receive power simultaneously from electrical power supply 202. Alternately, transducers 310 and 312 can be configured to receive power individually. In the embodiment illustrated in FIG. 23, the head portion 302′ includes a vibratory device 313 comprising an eccentric vibrator motor 313. Other types of vibratory devices do not depart from the scope of the present invention.

In the exemplary embodiment (FIG. 1), applicator 104 includes an imaging transducer 311 (e.g., a transceiver) for sending and receiving ultrasound suitable for imaging the treatment site. Processor 208 may be configured to process the ultrasound for imaging, as explained in more detail below.

In some embodiments, applicator 104 is configured to provide multiple treatment modalities to treat infection. In addition to providing ultrasound energy by operation of ultrasonic transducer 310, device 100 may be configured to apply additional energy, other than HFUS energy, to the treatment site. For example, applicator 104 may include additional treatment components 316 and/or 318. In one embodiment, treatment component 316 comprises a transducer configured to provide extracorporeal shockwaves to the treatment site and/or pulsed electromagnetic frequency (PEMF) energy to a treatment site. In one embodiment, component 318 is a conductor configured to provide electrical AC current in the radio frequency range or DC current. In yet another embodiment, the treatment component 316 may include a source of ultraviolet light for using in treating in conjunction with the ultrasonic transducer. The source of ultraviolet light may emit light in the C range from about 270 nanometers to approximately 320 nanometers.

To inhibit overheating of applicator 104, a temperature sensor 320 is coupled within applicator 104 to provide temperature feedback to processor 208. If the temperature sensed by temperature sensor 320 is greater than a threshold temperature, processor 208 may be configured to reduce intensity of the drive signal or discontinue treatment using device 100 until the temperature falls within an acceptable range. In the embodiment illustrated in FIG. 23, the head portion 302 includes a temperature sensor 320′ and a heat sink 321 to reduce overheating of the applicator 104. The heat sink 321 is in thermal contact with the transducer 310′ to transfer heat from the transducer to the heat sink to inhibit overheating of the shell 306′. The heat sink 321 may comprise any suitable thermally conductive material having a thermal conductivity greater than the shell 306′, for example.

In the illustrated embodiment, a transmission component 330 is coupled to head portion 302 of applicator 104. Transmission component 330 is fabricated to enable transmission of ultrasound energy from applicator 104 into the body of a subject without a coupling gel. In one embodiment, transmission component 330 is an overmold coupled on head portion 302. In the exemplary embodiment transmission component 330 is fabricated from silicone. Alternatively, transmission component 330 can be fabricated from any material that enables the transmission of energy from applicator 104 to a treatment site within the body of the subject including, but not limited to, an ultra-high-molecular-weight polyethylene, a thermoplastic elastomer, and polytetrafluoroethylene. In some embodiments, transmission component 330 is a reservoir that includes an aperture for inserting and extracting material in the reservoir. In such an embodiment, gels and/or other substances capable of transmitting energy from applicator 104 to a treatment site within the body can be heated or cooled before inserting into the reservoir to provide heating or cooling to tissue that contacts transmission component 330. A specific drain or aspirate can be provided in addition to the vibratory circuit.

In one embodiment, head portion 302 includes at least one aperture 340 in a portion of head portion 302 that contacts the skin of the user (e.g. transmission component 330). In such an embodiment, a suction component 342 may be coupled to the at least one aperture 340 to provide suction pressure and create a partial vacuum at the skin of a user to aid in retaining applicator 104 against the skin of a user during a treatment session.

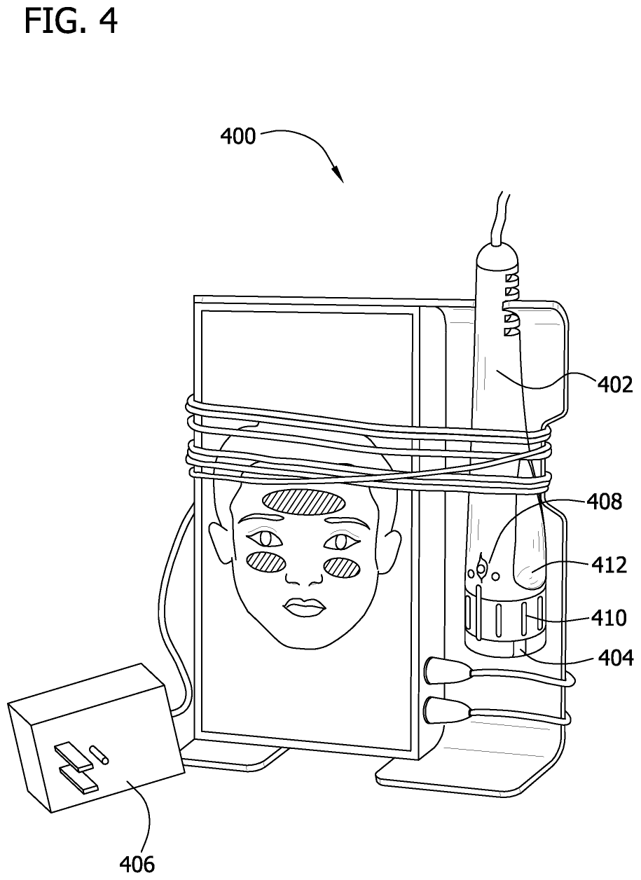

FIG. 4 is a perspective view of an alternative HFUS device 400 having the components of device 100 shown in FIG. 1. In the exemplary embodiment, the features of device 100 are integrated into a single handheld unit that is configured to treat infection within the subject. For example, device 400 includes a device housing 402, a treatment applicator 404, and a power source 406. Device housing 402 includes a user interface 408 similar to the first embodiment. In one embodiment, device 400 is configured to be hand-held and portable such that a user can utilize device 400 with one hand. In some embodiments, applicator 104 and/or housing 102 are configured to have an ergonomic design when held by a user, such as a design including one or more recesses 412 that aids in stabilization.

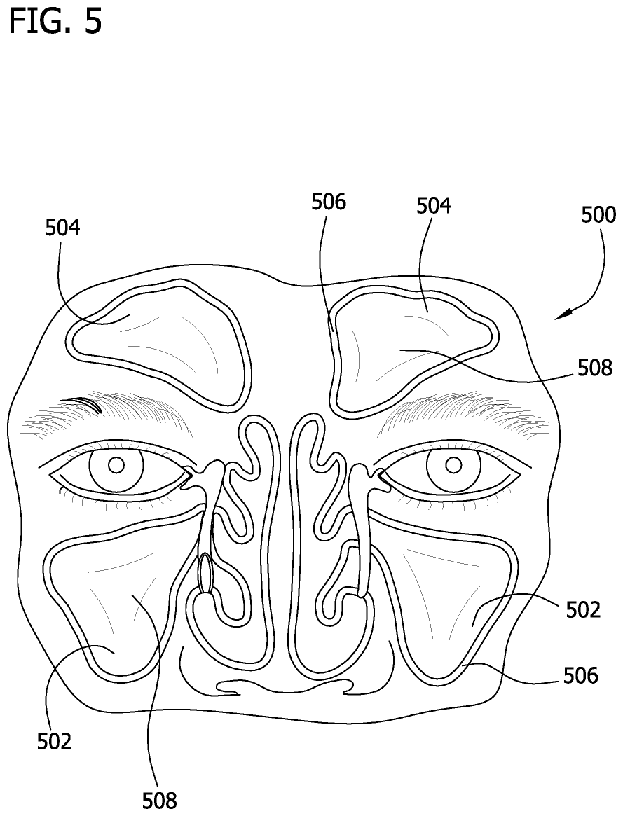

It should be noted that devices 100 and/or 400 are shown to be configured for treating infection in the sinus of a subject. Devices 100 and/or 400 are shown as being configured to treat treatment sites including the frontal 502 and maxillary sinus 508 (shown in FIG. 5) with the transducer delivering ultrasound energy through the skin and into the sinus cavity to treat infection.

High Frequency Ultrasound (HFUS)

To validate the effectiveness of treating infection with HFUS energy, testing of the output of device 100 shown in FIG. 1 was performed in a medical biofilms laboratory. As shown in more detail below, the output of device 100 was tested against (1) a methicillin-sensitive (MSSA) Staphylococcus aureus strain isolated from a sinus of a subject with chronic rhinosinusitis and (2) a methicillin-resistant (MRSA) Staphylococcus aureus strain isolated from a chronic wound of a subject. A CDC biofilm reactor (CDC-BR) was used to grow MSSA Staphylococcus aureus and MRSA Staphylococcus aureus biofilms on polycarbonate coupons that were subjected to testing.

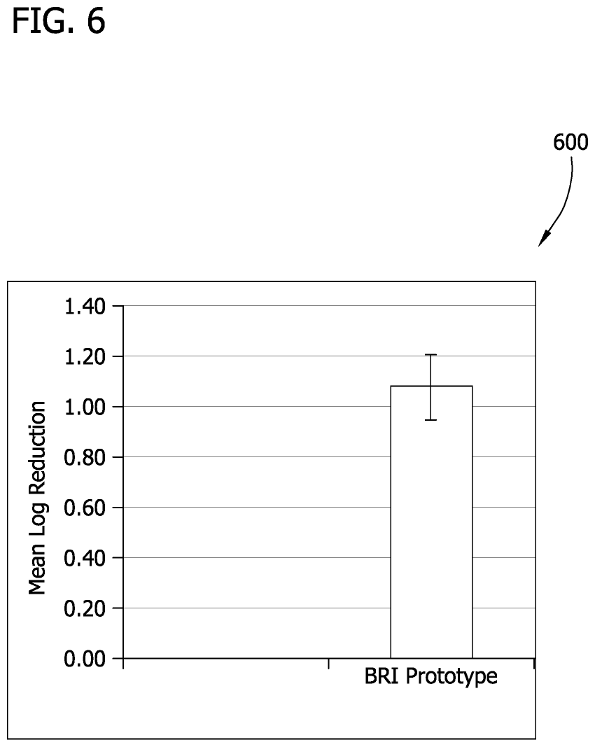

Test 1—HFUS energy from device 100 was tested on MSSA Staphylococcus aureus coupons. The mean log density (MLD, ±standard deviation) of the control biofilms was 7.95±0.05 log CFU/cm². Using device 100, coupons were exposed to five minutes of HFUS energy from applicator 104 at a frequency of 1 MHz and an intensity of approximately 1 W/cm². As shown by graph 600 in FIG. 6, treatment of the coupons with device 100 resulted in a mean log reduction (MLR) of (1.08±0.13) yielding a 91.41% reduction of MSSA Staphylococcus aureus biofilm on the coupons.

To calculate the elimination of bacteria and/or biofilm in Test 1, the treatments were assessed relative to untreated controls using viable plate count methods. The coupons were placed in tubes containing 10 ml phosphate-buffered saline (PBS). A sequence of vortex, sonicate, and vortex was then used to remove bacteria from the coupons and produce a bacterial suspension. The suspension was serially-diluted in PBS and plated on Tryptic Soy Agar (TSA). The plates were incubated at 37° C. for 24-48 hours and the number of colony forming units (CFU) was counted. Based on the dilution and the dimensions of the coupons, the CFU per unit area (CFU/cm²) was calculated. The CFU/cm² counts were logarithmically transformed (base 10) to determine log density (LD) and a mean log density (MLD) was calculated from replicate coupons.

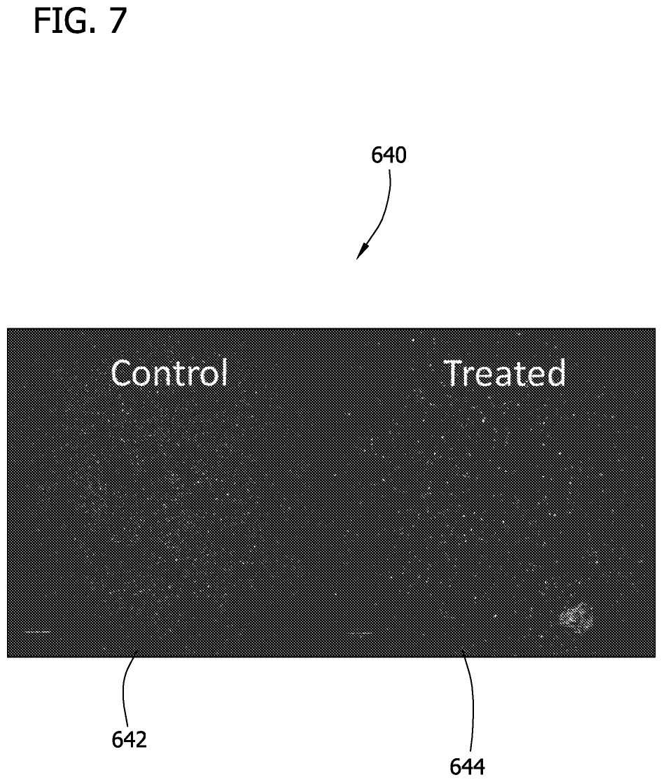

Test 2—FIG. 7 displays microscope images 640 of the results of Test 2 using device 100 shown in FIG. 1. For Test 2, MSSA Staphylococcus aureus biofilms were grown in the CDC-BR, as described above, and the coupons were subjected to HFUS output from device 100 with a power level of 1 W/cm² (100%) at 1 MHz for 5 minutes. Two coupons were treated and one coupon served as an untreated control. After treatment, the coupons were treated with the LIVE/DEAD® BacLight™ Viability Kit which includes two nucleic acid stains, SYTO-9 and propidium iodide. SYTO-9 stains live bacterial cells green and propidium iodide stains bacterial cells with damaged membranes (dead cells) red. After treatment of the viability kit, coupons were imaged with a Leica SP5 confocal scanning laser microscope. As is shown by pictures 640, the coupon subjected to HFUS output 644 from device 100 had less infectious agents (e.g. bacteria and/or biofilm) than the control coupon 642.

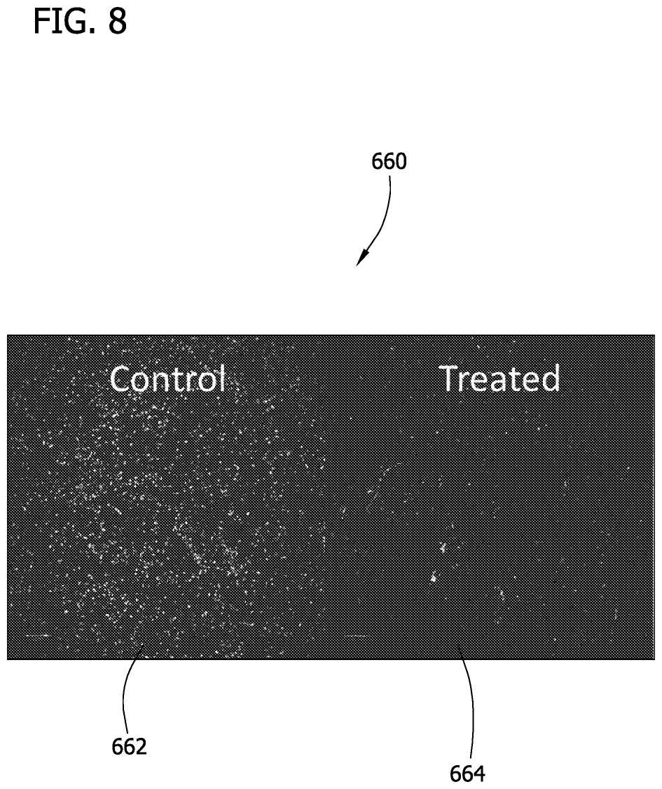

Test 3—FIG. 8 displays microscope images 660 of the results of Test 3 using device 100 shown in FIG. 1. For Test 3, MRSA Staphylococcus aureus biofilms were grown in the CDC-BR, as described above, and the coupons were subjected to HFUS output from device 100 with a power level of 1 W/cm² (100%) at 1 MHz for 5 minutes. Two coupons were treated and one coupon served as an untreated control. After treatment, the coupons were treated with the LIVE/DEAD® BacLight™ Viability Kit which includes two nucleic acid stains, SYTO-9 and propidium iodide. SYTO-9 stains live bacterial cells green and propidium iodide stains bacterial cells with damaged membranes (dead cells) red. After treatment of the viability kit, coupons were imaged with a Leica SP5 confocal scanning laser microscope. As is shown by pictures 660, the coupon subjected to HFUS output 664 from device 100 had less infectious material (e.g. bacteria and/or biofilm) than the control coupon 662. As shown by the results of Tests 1-3, shown in FIGS. 6-8, device 100 is configured to negatively affect infectious agents inside the body. As described above and illustrated in FIGS. 6-8, the systems and methods described herein enable a user to treat biofilm. It is believed the output of ultrasonic vibratory energy at a frequency of 1 MHz has an effect on infectious agents and/or biofilms by turning infectious agents, such as bacteria, and/or biofilm into planktonic state, causing delamination of biofilm, creating a physical disruption, and/or breakdown of polysaccharides present in biofilm matrix.

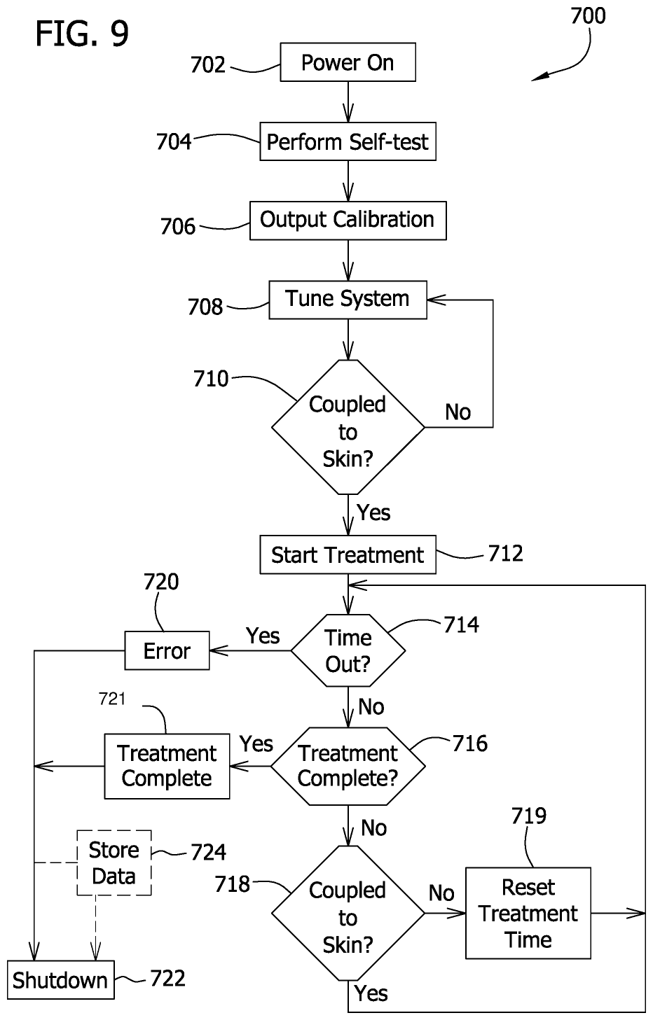

FIG. 9 is an exemplary flowchart of a method 700 for use with device 100 shown in FIG. 1. To initiate method 700, device 100 receives (e.g., at the power on step 702) instructions to start a treatment session from input interface 110 and/or presentation interface 108. Alternatively, treatment information can be transmitted in any known manner including through communication interface 108. In some embodiments, treatment information relates to a particular body area and/or region of the body to be treated. Alternatively, treatment information relates to the specific infection to be treated. After receiving instructions at step 702, device 100 performs a self-test at step 704. It should be noted that power and error checking algorithms performed during the self-test of step 704 can be implemented at any time throughout method 700. In the exemplary embodiment, processor 208 communicates with all hardware components to determine if any errors occur.

After performing a self-test at step 704, processor 208 calibrates the output of applicator 104 (step 706). In the exemplary embodiment, processor 208 tunes device 100 at step 708, based on the received treatment information. In one embodiment, tuning of device 100 is performed by determining a resonance (e.g. parallel or series) and locking into a frequency at the resonance selected. In one embodiment, tuning of device 100 at step 708 is performed when temperature sensor 320 detects a temperature that exceeds a predetermined threshold for applicator 104. Once device 100 is tuned (step 708), device 100 determines at decision block 710 if applicator 104 is coupled the skin of a user. To determine whether the applicator 104 is coupled to the user at 710, device 100 compares the impedance feedback received to a threshold that is correlated to applicator 104. In one embodiment, the impedance of applicator 104 when the applicator is coupled to skin is in the range of 100-500 ohms. Alternatively, the impedance range can be any range that correlates to the properties of the applicator 104.

After determining applicator 104 is coupled to the skin (decision block 710), device 100 starts a treatment session at step 712 and outputs energy (e.g. HFUS and tactile vibratory energy) through applicator 104 based on the received 702 treatment information. During the treatment session, processor 208 monitors device 100 to determine if device 100 has timed out (decision block 714), completed a treatment time (decision block 716), and/or determined that applicator 104 has been continuously coupled to the skin (decision block 718). It should be noted that processor 108 continuously determines if applicator 104 is coupled to the skin in the same manner as described in the determination step 710. If during a treatment session processor 108 determines that applicator 104 is not coupled to the skin, the treatment time is reset at step 719. The transducer may be held in position or signals could be applied to a treatment site for a few milliseconds, few seconds, or a few minutes before moving the transducer to a new location or altering the signals for application at a different treatment site. This treatment protocol could be done robotically. The device 100 could be used with timers by holding the applicator 104 at one position for a period of time and then moving it to another position. It could be simply isolated and rotated so that the ultrasonic frequencies or energy would be dispersed over a larger surface area through rotational movement. The output could be variable or continuous pulse. The output could be mobile, robotically positioned, or sequentially positioned for a time, distance, or specific angle location. This could be varied either remotely, via robot, or via control.

If processor 208 determines at decision block 714 a timeout has occurred, an error is provided to a user at step 720, device 100 is shutdown at step 722, and output through applicator 104 is stopped. Additionally, if processor 208 determines at decision block 716 that a treatment session is complete, the user is alerted that the treatment is complete at step 721 and device 100 is shutdown at step 722. In the exemplary embodiment, an alert is provided to a user visually (e.g. blinking light or changing light color) and/or audibly when an error occurs at step 720 or the treatment is complete at step 721. Alternatively, alerts at steps 720 and 722 can be provided to the user in any manner that facilitates notification to a user. In some embodiments, before device 100 is shutdown at step 722, all treatment session data is stored at step 724 in memory device 209 for transferring to another device.

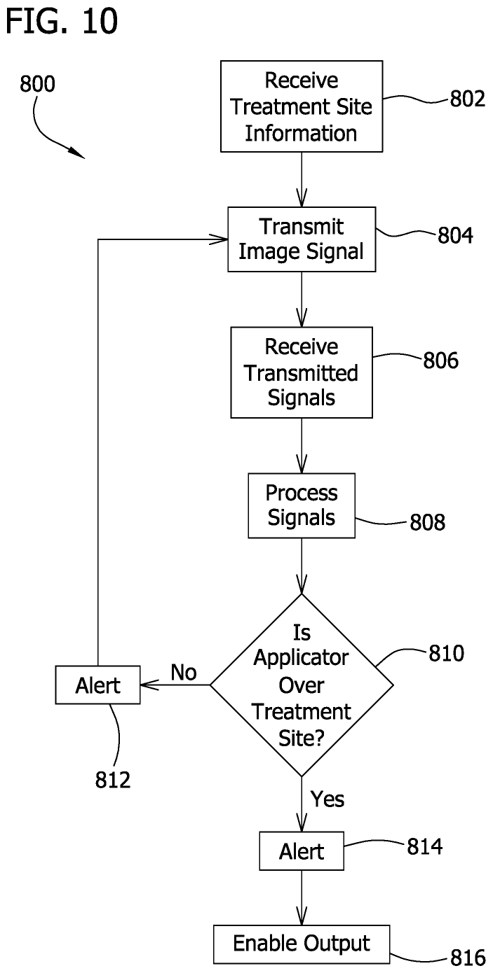

FIG. 10 is an exemplary flowchart of a method 800 for determining a location of device 100, shown in FIG. 1, in relation to a treatment site. To determine a location of device 100, a user selects a treatment site, such as by touching a graphical representation of the treatment site on display, and the selection is received at step 802. In the exemplary embodiment, a treatment site is selected through user interface 108 and transmitted to processor 208. The treatment site can be anywhere inside the body including, but not limited to, sinuses 502 and 504. Once the treatment site selection is received at step 802, imaging signals from imaging transducer 311 are transmitted at step 804 through applicator 104. In the exemplary embodiment, the imaging signals are pulsed ultrasound. Alternatively, the imaging signals can be any imaging signal that enables locating device 100 as described herein.

When imaging signals are transmitted in the body at step 804, they are reflected from objects (e.g. tissue) in the body, and are received by applicator 104 at step 806. In one embodiment, imaging transducer in applicator 104 transmits and receives the imaging signals described herein. Alternatively, the imaging signals are transmitted and received by a transducer in an array of transducers positioned in applicator 104.

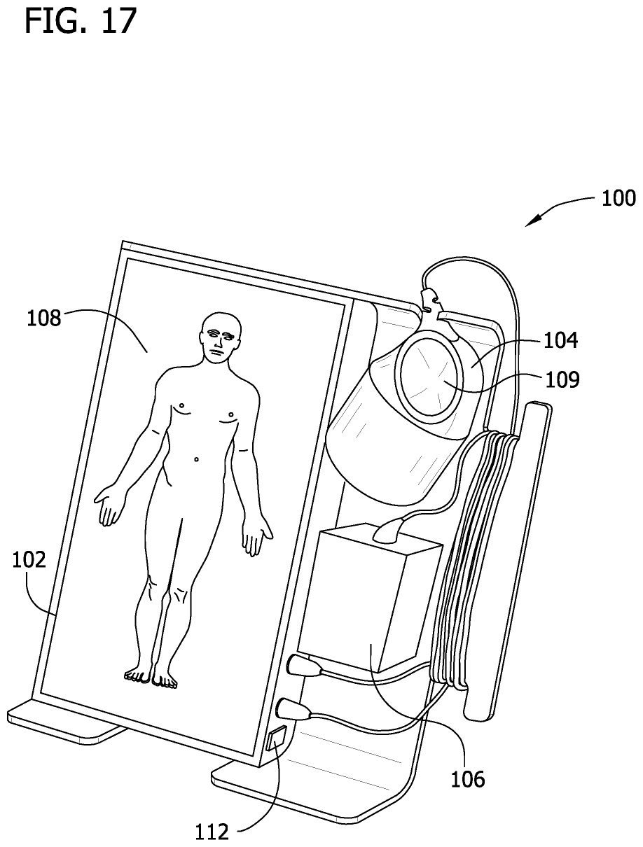

When the transmitted imaging signals have been received at step 806, the signals are processed by processor 208 at step 808. In the exemplary embodiment, the processed signals determine patterns of objects in the body and/or distances of the objects inside the body. The processed signals are then compared at decision block 810 to known patterns and/or distances of the received treatment site to determine if applicator 104 is over the treatment site. In some embodiments, processor 208 performs the comparison 810 by determining if the processed signals are within a predetermined threshold of known and/or stored information about the treatment site. As shown in FIG. 17, processor 208 may be configured to generate a graphical image of a body and indicate on the graphical image the location of applicator 104.

If processor determines at decision block 810 that applicator 104 is not over the selected treatment site, the user is alerted at step 812 and image signals are transmitted at step 804 again. If processor determines at decision block 810 that applicator 104 is over the selected treatment site, the user is alerted at step 814 and treatment modalities of device 100 are enabled to be output at step 816. In the exemplary embodiment, alerts at steps 812 and 814 are provided to the user through user interface 108. In the exemplary embodiment, alerts at steps 812 and 814 are visual. Alternatively, alerts at steps 812 and 814 can be communicated to a user in any manner that facilitates notification as described herein including, but not limited to, auditory signals and/or tactile feedback sent from device 100. In one embodiment, the step of sending the alert 812 includes providing the user the determined location of applicator 104. For example, if a user selects the knee as a treatment site and the applicator is positioned over the tibia, the user would visually see that the applicator is not over the selected treatment site and that it is on the lower leg. It should be noted that method 800 could be utilized anywhere throughout method 700 shown in FIG. 10. In another embodiment, a marker (i.e., a detectable device) may be provided on an implant or within a desired treatment site. The marker may be detectable by device 100. For example, the marker may be an RFID tag or magnetic tag or other component detectable by a sensor. Device 100 may include a detector for detecting the marker to determine if applicator 104 is correctly positioned for treating the treatment site. In one example, the marker may be biodegradable and/or degradable based on use and treatment. For example, the marker may be degradable by ultrasound such that after the marker is subjected to a certain amount of ultrasonic treatment using device 100, the marker is no longer detectable by the device. In this example, the degradation of the marker signifies that treatment has been completed.

The methods and systems described herein can be utilized to treat infections anywhere inside the body. In one embodiment, the methods and systems described herein are utilized to treat sinusitis. In one example, device 100 provides treatment of infection in the sinus using applicator 104 with method 800 by treating the frontal sinus 502 with ultrasound energy having a frequency of 1 MHz and an intensity of 0.5 W/cm² for 2 minutes, and the maxillary sinus 504 with ultrasound energy having a frequency of 1 MHz and an intensity of 1.0 W/cm² for 2 minutes. The treatment time, frequency and power of the applied ultrasound energy can vary depending on specific types of infections.

In one embodiment, method 700 and/or 800 can be utilized to determine if there has been a buildup of fluid in and/or around a portion the body, such as sinus 500 shown in FIG. 5. For example, during acute and chronic sinusitis there is a buildup of fluid in sinus cavity 502 and/or 504. When a sinus cavity is void of fluid, the imaging ultrasound signal will reflect off of an anterior side 506 of the cavity and not propagate due to the air in the cavity. When there is infection (e.g. sinusitis), which often leads to the presence of fluid (e.g. mucus), the fluid will allow the imaging signal to propagate to a posterior wall 508 of the sinus cavity and reflect an echo. In such an embodiment, device 100 can be optimized to compensate for the fluid in the sinus cavity 500 and provide output energy that will be transmitted to both sides 506 and 508 of the sinus cavity 500. To accomplish this, as described above, separate transducers with varying signals or components of a transducer array could be utilized.

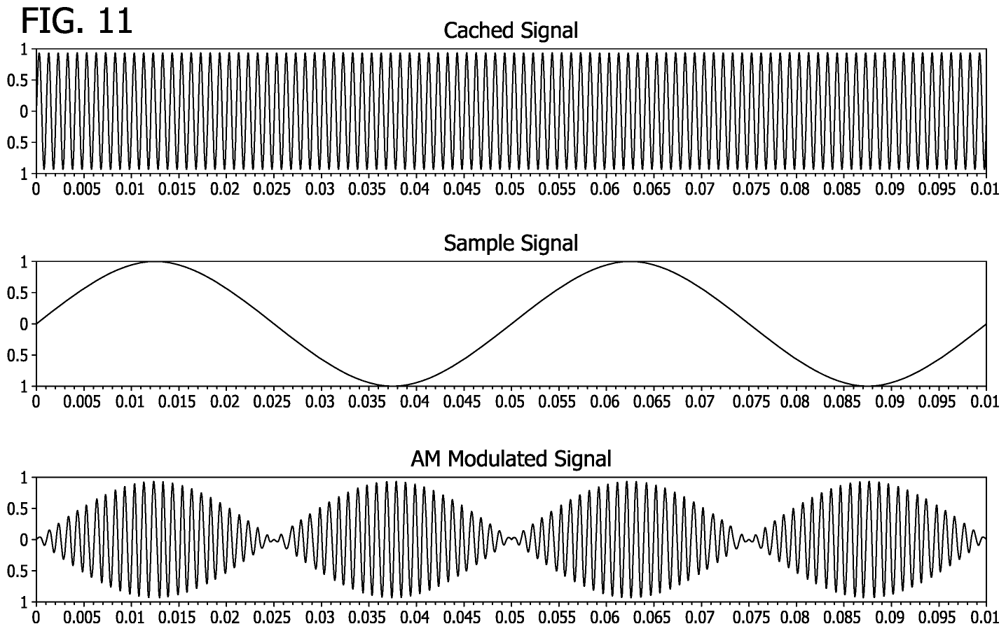

In some embodiments, the HFUS energy from applicator 104 is modulated. In such an embodiment, a narrow beam of ultrasound (carrier) is amplitude modulated (AM) with an audio signal which creates a narrow beam that can only be heard along the path of the beam, or from objects in the path of the beam. Air has non-linear acoustic properties that cause the signal to self-demodulate over the path of the beam (shown in FIG. 11).

The concept of self-demodulating AM signals from non-linearities in the transmission media can be applied to the problem of getting optimal LFUS frequencies for biofilm and bacteria reduction to the sinus cavities via HFUS waveforms. As ultrasound travels through different materials, the wavelength (λ) is determined by the speed of sound (c) through the media the waveform is traveling through divided by the frequency of the waveform (f), as shown by the following equation:

λ=c/f

The speed of sound (c) for various materials is shown in the table below.

| Material | Velocity (m/s) |
|---|---|
| air | 331 |
| fat | 1450 |
| water (50 C) | 1540 |
| human soft tissue | 1540 |
| brain | 1541 |
| liver | 1549 |
| kidney | 1561 |
| blood | 1570 |
| muscle | 1585 |
| lens of eye | 1620 |
| skull-bone | 4080 |
| brass | 4490 |
| aluminum | 6400 |

By using a waveform that is optimized for bacteria and biofilm removal and taking advantage of the non-linearities caused by the change in the speed of sound at the interface of different biologic materials, device 100, and more specifically transducer 310, is configured to treat infections with optimal waveforms, as well as manage the pain and discomfort associated with the condition while ensuring patient safety during the treatment, using the information below. To ensure patient safety and comfort, the carrier signal is selected to be above 20 kHz, with the optimal frequencies being between 1 MHz-3 MHz.

In one embodiment, the algorithm utilized for treatment modulates the treatment signal over a 1-second (1000 mS) period. If pulsed ultrasound treatment (PUS) is used to minimize heating, a typical pulse ratio is 1:9, meaning the ultrasound output would be active for 1 ms and off for 9 ms. Each pulsed cycle would take 10 ms.

$$\frac{1000 mS}{10 mS} = 100 frequency steps$$

Assuming the desired frequency range for the treatment signal is 20 kHz-80 kHz, the frequency step would be calculated as follows:

$$\frac{80 kHz - 20 kHz}{100 steps} = 600 Hz / Step$$

In this example, the carrier frequency would be Hz 1 MHz, and the treatment signal would start at 20 kHz. After every pulse cycle of 10 ms, the frequency of the treatment signal would be increased by 600 Hz. Once the upper limit of the treatment signal frequency is reached, in this example 80 kHz, the algorithm would be repeated starting at the start frequency until the desired total treatment time is reached. In one embodiment, the acoustic output will have a resonant frequency at 1 MHz and the AM envelope at a frequency=20 kHz+n≈600 Hz. Where n=loop count, and n<100.

With this algorithm, each selected treatment envelope frequency is output for the same length time, but the actual number of periodic waveforms would vary with the frequency being used. At the lowest treatment frequency, 20 kHz, each envelope period is 0.05 ms. This gives 20 periods of this modulated treatment signal over this (0.1 ms) frequency step. At the highest selected treatment frequency, 80 kHz, each period is 0.0125 ms, giving 80 period of the modulated treatment signal over this (0.1 ms) frequency step. The algorithm could be adjusted so that each desired treatment frequency is active for the same number of periods of the modulated signal, instead of the same amount of time. It is contemplated that the output could be continuous ultrasound (CUS) instead of PUS.

In one embodiment, the algorithm is modified so that the amount of ultrasound envelope determines the length of the treatment envelope, rather than a predetermined time. In cases where the user is applying a coupling gel to the surface of the skin prior to treatment, it is contemplated that once applicator 104 is in contact with the treatment site, the amplitude of the carrier frequency could be gradually increased prior to treatment to allow bubbles in the coupling gel to degas or dissipate. It is also considered that suction, or negative pressure, could be used to achieve better coupling of applicator 104 with the skin.

In one embodiment, a simple graphic on the presentation interface 104 indicates the area to be treated. For example, a picture of a face could be shown with sinus areas capable of illumination. If the setting for the frontal sinuses is selected, LEDs behind the frontal sinus area would illuminate. If the maxillary sinus is selected, LEDs behind the maxillary sinus area would illuminate. The optimal time, power, and modulation schemes would be selected based on the area to be treated. The display could also be implemented with an LED, VFD, or other methods known in the art.

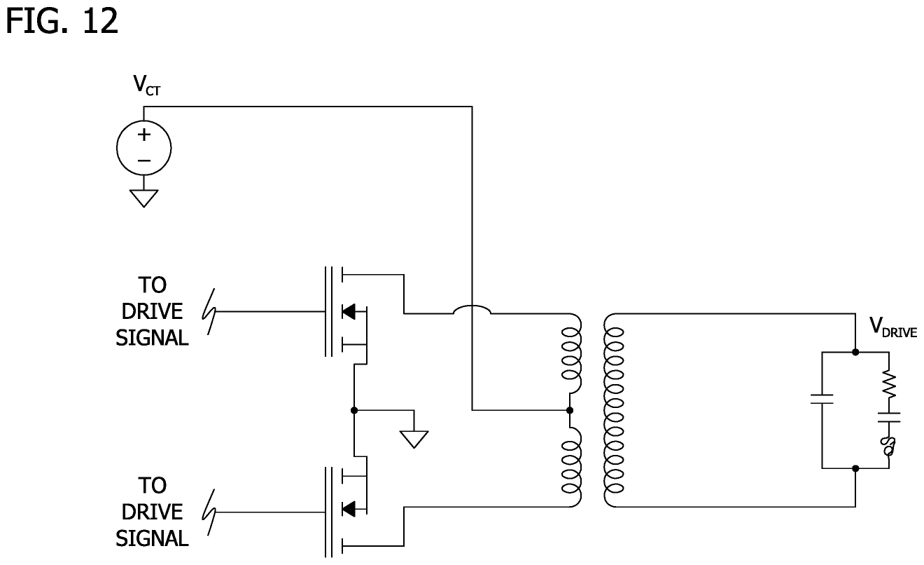

Referring to FIG. 12, a traditional center tapped transformer in a push pull configuration can be used to implement aspects of a modulation scheme. The voltage to the center tap could be configured to oscillate at the target treatment frequency while the carrier frequency would be pulsed to the push-pull FETs. Other analog and digital methods of creating the modulated signal which are known in the art could be implemented as well.

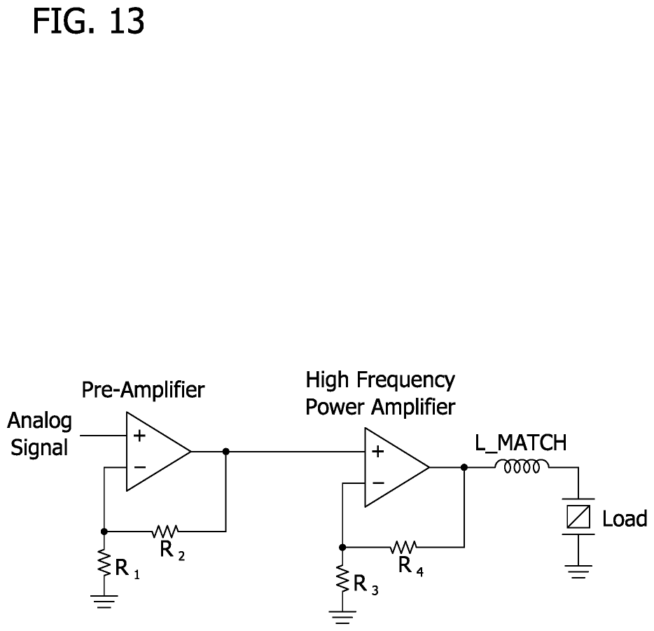

Referring to FIG. 13, in another embodiment, a waveform generator and high frequency power amplifier configuration implements the modulation. The modulated signal, possibly supplied by a processor, would be fed into the pre-amplifier with a predetermined gain of Av=1+(R2/R1). The amplified signal would then feed into the high frequency power amplifier, which would amplify the signal again with the predetermined gain of Av=1+(R4/R3). When driving a piezoelectric transducer, a matching inductor is added in series to the load to compensate for the capacitance of the load.

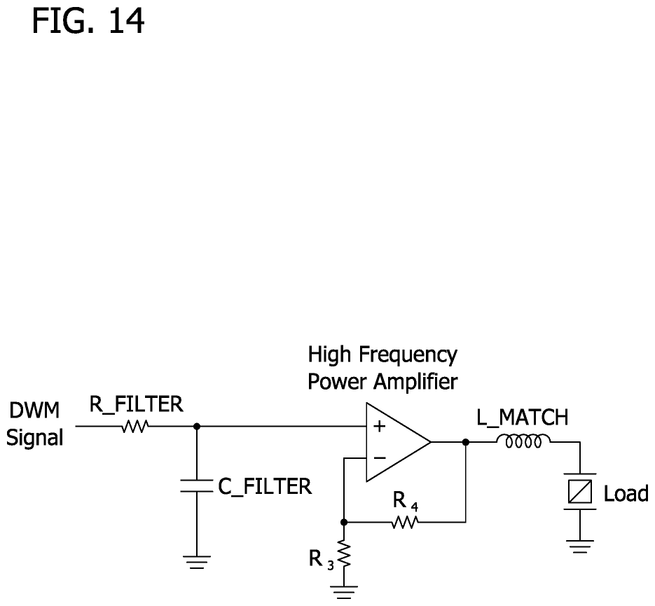

Referring to FIG. 14, in another embodiment, a pulse width modulated signal and high frequency power amplifier configuration implements aspects of the modulation (shown in FIG. 14). A pulse width modulated signal, possibly supplied by a processor, is fed into an RC filter. The RC filter acts as a digital to analog convertor and convert the pulse width modulated signal into an analog signal. The cut-off frequency of the RC filter is determined by the equation Fc=1/(2*Pi*R*C). The converted analog signal is then fed into the high frequency power amplifier with a predetermined gain of Av=1+(R4/R3). When driving a piezoelectric transducer, a matching inductor is added in series to the load to compensate for the capacitance of the load.

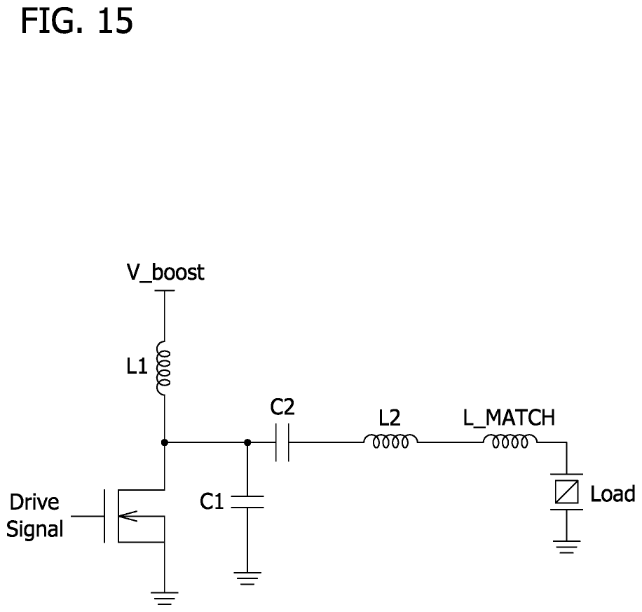

In another embodiment illustrated in FIG. 15, a Class E amplifier is used to implement aspects of the modulation. The Class E amplifier is suitably designed specifically for the drive frequency and N-Channel Mosfet that is defined for the system. The drive frequency is determined by the desired frequency to drive the load. The values of R_Load, L1, C1, C2 and L2 are determined using the following equations:

$${R}_{Load} = \text{?} = 0.576801 - ( 1.0000086 - \text{?} - \text{?} + \text{?} )$$

$$C 1 = \frac{1}{34.2219 * f * R} * ( 0.99866 + \frac{0.91424}{QL} - \frac{1.03175}{{QL}^{2}} ) + \text{?}$$

$$C 2 = \frac{1}{2 * Pl * f * R} * ( \frac{1}{QL - 0.104823} ) ( 1.00121 + \frac{1.01468}{QL - 1.7879} - \text{?} )$$

$$L 2 = \frac{QL * R}{2 * Pl * f}$$

$$\text{?} \text{indicates text missing or illegible when filed}$$

When driving a piezoelectric transducer, a matching inductor is added in series to the load to compensate for the capacitance of the load.

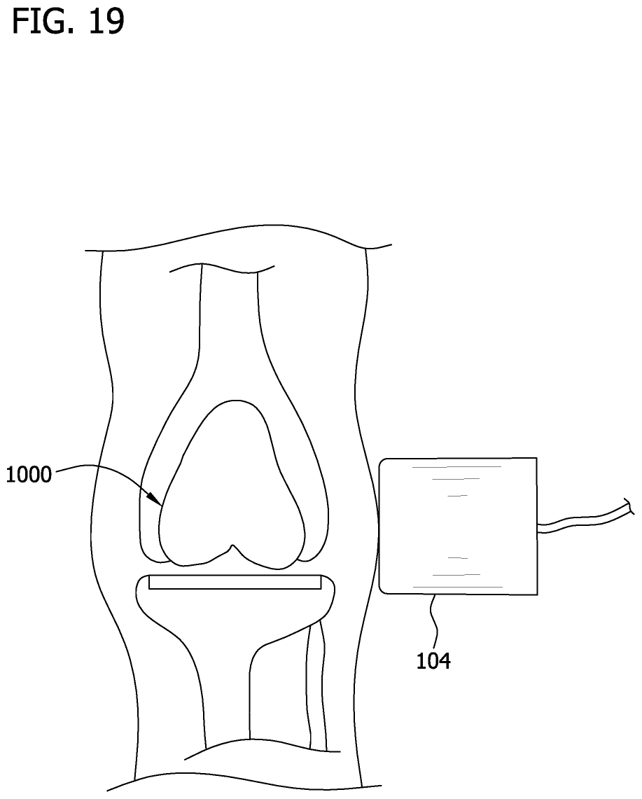

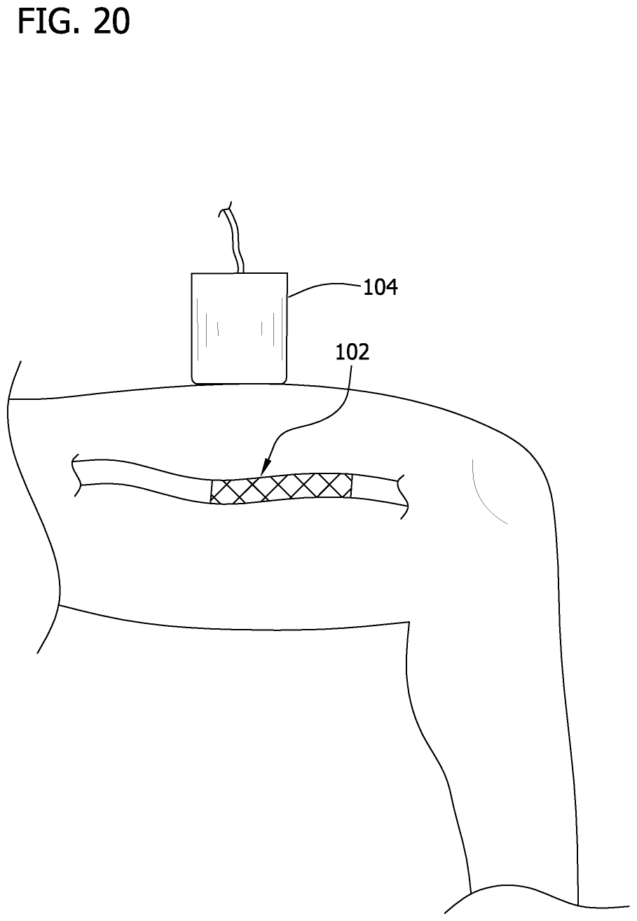

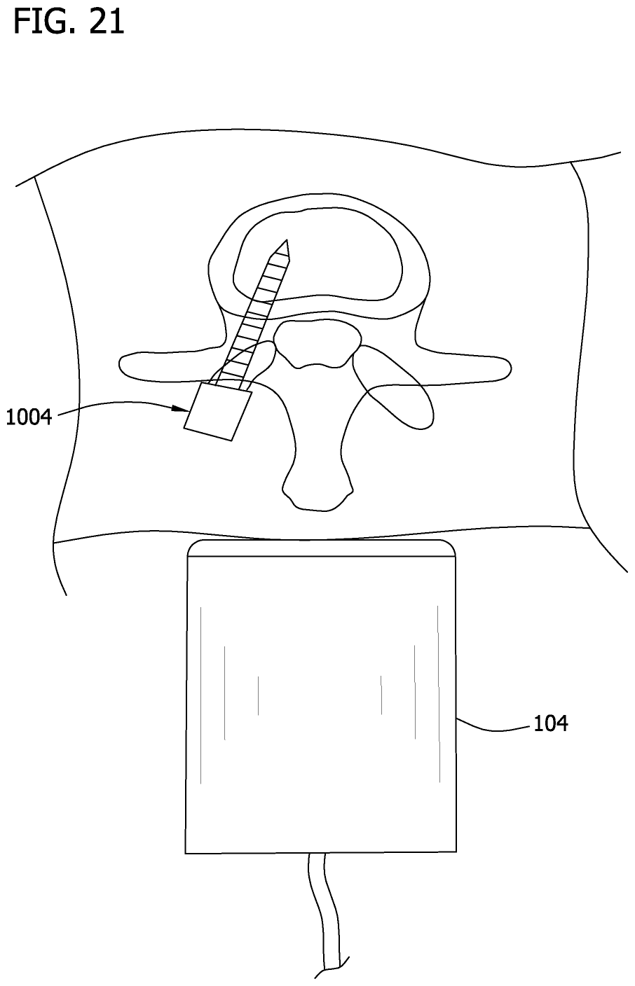

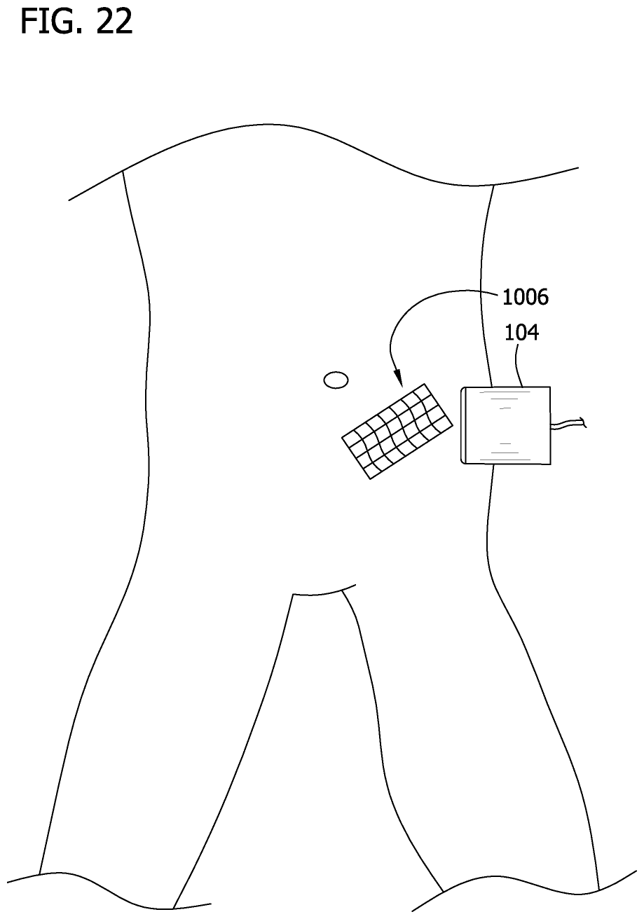

While some methods and systems have been described in treating infection in the sinus, it is also contemplated that the methods and systems described herein could be used to treat infection in other sub-dermal and dermal treatment sites including, but not being limited to, post-operative sites, joint replacements, metallic implants, polymeric implants, cystic fibrosis, skin ulcers, on and/or around the ear (e.g. otitis), infected nails, endothelium, vascular, or any passageway (i.e. bronchi, joint space synovium or intraarticular space, peritoneum, pleura, or prostate). For example, as shown in FIG. 19, device 100 can be used to treat infection for a knee replacement 1000 implanted in a subject's body. The components of knee replacement 1000 for treatment by device 100 may include metal components, polymeric components, biologic components, and mesh components, among other types of components. In another example, shown in FIG. 20, device 100 can be used to treat infection at a stent 1002 (e.g., peripheral stent, as shown, or coronary stent, or neurovascular stent) implanted in a subject's body. The components of stent 1002 for treatment by device 100 may include metal components, polymeric components, biologic components, and mesh components, among other types of components. In yet another example, shown in FIG. 21, device 100 can be used to treat infection at a screw 1004 (e.g., spinal screw, as illustrated, or other fastener) implanted in a subject's body. The components of screw 1004 for treatment by device 100 may include metal components, polymeric components, biologic components, and mesh components, among other types of components. In yet another example, shown in FIG. 22, device 100 can be used to treat infection at a mesh 1006 implanted in a subject's body. The components of mesh 1006 for treatment by device 100 may include metal components, polymeric components, biologic components, and mesh components, among other types of components.

In one embodiment, device 100 is utilized to prevent capsular contracture. Capsular contractures occur when the collagen-fiber capsule formed around a foreign material implanted in the human body tightens and is squeezed together. The foreign material can include, but is not limited to, breast implants, artificial pacemakers, and orthopedic prostheses. It has been shown that bacterial biofilms on foreign material implanted in the body may cause chronic inflammation and contribute to this condition. Device 100 can be utilized to prevent the capsular contracture by treating biofilm, treating scar tissue, and increasing blood flow.

Included within the treatment of biofilms are methods to prevent the formation biofilms. This could be done by using device 100 in the hand held form, or by integrating the device into existing medical devices such as surgical dressings, wraps, continuous passive motion devices, wound vacs, and adhesive bandages. In such an embodiment, applicator can be fabricated to have at least a portion that is flexible.

In one embodiment, local pressure is combined with the output of device 100 to enhance the effect. In such an embodiment, device 100 is configured to be inserted into and/or a portion of device 100 (e.g. applicator 104 or transducers) is configured to be integrated into a device that applies manual pressure or a compressive force (e.g., a blood pressure cuff). In the exemplary embodiment, a device that applies manual pressure is an orthosis for joint rehabilitation, including but not limited to, an orthosis from Joint Active Systems of Effingham, Ill. Devices may include a pressure sensor which would guarantee that a correct pressure is applied to external or internal applicators or transducers. The manual or compressive force would stabilize the device 100 against dynamic or pulsatile movement of the body tissue, which could affect location or position of the applicator or transducer.

Non-linearities and impedance mismatches can also be introduced into the body by injecting contrast agents, micro bubbles, fluids, and/or air. Device 100 can be utilized to apply ultrasound and micro bubbles to prevent the formation of scar tissues. In one embodiment, device 100 is configured to enhance and/or eliminate the use of micro-bubbles. By taking advantage of an acoustic mismatch or non-linearity at the interface of the bubbles, the signal could be modulated to enhance the therapeutic action of the micro bubbles. If there was enough of a mismatch at the interface at the area of targeted scar tissue, the use of micro-bubbles might not be required.

The acoustic mismatch described above can enhance the delivery of pharmaceuticals. In one embodiment, the methods and systems described herein are optimized for the selective rupture of cell membranes to enhance pharmaceutical efficacy. Pharmaceuticals could also be designed to have a specific resonance which could be excited to enhance delivery or cell rupture. The pharmaceuticals might be engineered to provide a large acoustic impedance mismatch. The signal could also be optimized to enhance the movement of pharmaceuticals across the blood brain barrier.

To aid in the disbursement and/or absorption of pharmaceuticals to increase a pharmaceutical's efficacy, the ultrasound output of device 100 can heat tissue as a result of vibratory energy. This heating can allow cellular absorption of pharmaceuticals to increase. Additionally, removal and/or elimination of a portion or all of bacteria and/or biofilm in a location will affect the pH of the tissue in and around the treatment site. As such, the changing pH of the tissue can enable increased absorption of pharmaceuticals. This could be used when delivering chemotherapy, vasodilators, bronchodilators, and hyaluronic acid. Accordingly, the device 100 may include one or more modalities for specific use in combination with pharmaceutical treatment to enhance the pharmaceutical treatment. The modality is configured to output desired ultrasound to increase a pharmaceutical's efficacy. As an example, the cell membrane and cell wall are rigid structures which prevent certain pharmaceuticals from passing into the cell (e.g., virus, parasites, and/or bacteria). For example, methicillin-resistant staph may be bacteria that function to resist drug effects by altering the cell wall/membranes of a non-resistant staph to prevent a drug from getting through the membrane. The present device 100 may include one or more modalities for delivering ultrasound configured to make the cell wall/membrane more porous so that drugs can easily enter into the cell to enhance treatment. The ultrasound may be applied in conjunction with adjusting the local pH to make a more alkaline environment. For example, a more alkaline environment may make the pharmaceutical agent more effective, especially in combination with ultrasound treatment.

In yet other embodiments, the device may include modalities for enhancing the effectiveness of other treatments, including but not limited to chemotherapy, stem cell therapy, biologic cell therapy, enzyme therapy, grafts (e.g., allograft and autograft), and biopharmaceuticals. Each modality may be specific to a certain additional treatment to produce a synergistic effect. That is, each modality is configured to output desired ultrasound that enhances the effectiveness of the particular treatment. For example, device 100 may be configured to deliver ultrasound to enhance tissue ingrowth or biological response to a graft (e.g., cartilage graft or cell graft). In a particular example, ultrasound from device 100 may be delivered to a scaffold made in accordance with U.S. Pat. No. 7,299,805, the entirety of which is incorporated by reference herein, to enhance the scaffold ingrowth fixation or stability. Ultrasound from device 100 could be used during the time of implantation to inhibit infection or enhance ingrowth due to its improved vascular function, and it could also enhance the pharmaceutical effects locally.

Device 100 may include one or more modalities for improving fluid flow of medicinal agents (e.g., pharmaceuticals, biologics, enzymes, etc.) into cells. For example, a modality of device 100 may be configured to make viscous fluid less viscous, thereby affecting cell walls or cell membranes, for example, by making previously resistant cell walls porous to medicinal agents. The agents may become more sensitive due to the fact the cell walls or cell membranes may be porous.

The systems and methods described herein can also be utilized with modalities to clean and/or eradicate residual debris resulting from the effects of the output of device 100. For example, a pulsed lavage can be used after treatment with device 100 to clean debris. In the example of sinus treatment, a user could utilize a nasal spray before, during, or after treatment of device 100 on a sinus treatment site. The spray would allow mucus positioned in the nasal cavities to escape quickly.

It is also thought that the parasitic diseases such as trichinosis, scabies, and toxoplasmosis could be treated with the methods and systems described herein. These parasites create spores, which are very resilient and difficult to treat. It is thought that these types of parasitic diseases may be treated by targeting the resonance of the spores and/or the acoustical impedance mismatch from the spore to the surrounding materials. Thus, the waveform can be optimized to break up the spores so they can be treated with pharmaceutical agents.

The methods and systems described herein may be used to optimize flow of fluid media by selecting a modulation frequency that is optimized to the media targeted for manipulation. This could be used for in-vivo applications such as blood flow in vessels or arteries. In one example, methods and systems could also be used for myocardial infarction to improve blood flow through an artery, for example, and/or to produce more laminar blood flow. If there is disruption or spasticity, ultrasound from the device 100 could be delivered to the location of the disruption that causes a heart attack or the location of a vasospasm. In another example, with respect to blood flow, the device 100 may include one or more modalities for increasing O₂, white blood cells, and/or nutrients to tissue. Ultrasound delivered by device 100 may enhance the body's normal response. A modality of device 100 may also make greater vascular permeability to allow greater oxygen tension, greater white blood cell delivery, and blood flow to specific areas. It can dilate the blood vessel as well. For example, a modality of device 100 causes delivery of ultrasound that increases blood flow or vascular dilatation or vascular permeability to deliver more nutrients, white blood cells, and oxygen, which enhances the normal body response and its efficacy. Depending on the configuration of the waveform, it could be used to cause or prevent constriction or spasms in the vessels. In one embodiment, device 100 is utilized to heat mucus in the body enabling the mucus to flow.

An ultrasound waveform could also be used in medical devices and instrumentation to enhance the flow of fluids and pharmaceuticals. Also industrial applications such as injection molding, oil pipelines, gasoline and biofuel production and transport could benefit. This could be done with injection molding for fracking of oil/gas with or without agents to enhance the viscosity of gas, oil, etc. Ultrasound could be applied to move, suction, or break up debris at the fracking tip so that fluid would flow rather than be obstructed by material, sand, etc. The methods and systems described herein could be used in conjunction with the drag modulation techniques described in U.S. Pat. No. 6,842,108 to Peter M. Bonutti, the contents of which are incorporated by reference herein in their entirety. Combining elements of the techniques enables a more efficient reduction of drag, fluid flow, and acoustic disturbance. The device 100 could be used to eliminate bubbles, separate particles from liquid, and/or further pack or compress materials as needed. This could be used to separate materials of different viscosities (e.g., oil, water, polymer metal or different types of metals). The device could be used for molding and manufacturing as well as inside the human body.

The systems and methods described herein can be used with mechanical agents, such as pseudoephedrine or other vasodilators or water. Moreover, the systems and methods may be used to enhance delivery of Cannabidiol (CBD) and Tetrahydrocannabinol (THC) for pain via sonophoresis. The device 100 may increase the transdermal absorption of semisolid topical compounds. This method of delivery will allow localized delivery of the medication, potentially eliminating the need for systemic delivery. This method could potentially maximize the effectiveness of pain treatment while minimizing the psychoactive effects. The method may include the use of vasodilators, solvents, thermal optimization, and/or pH optimization to enhance efficacy.

The acoustic mismatch between biologics in the body may allow for the separation of (e.g., delamination of) biologic and/or non-biologic materials throughout the body based on the modulation scheme that is selected. This could allow for acoustical separation of dissimilar materials including but not limited to mucus, calcium deposits from vascular system or articular cartilage, earwax, or the treatment of chondrocalcinosis. As such, varying frequencies can be transmitted into a treatment site to affect attachment to different surfaces. For example, for use after a knee arthroplasty, device 100 could transmit a first frequency to affect bacteria or biofilm attachment to a native surface (i.e. bone or soft tissue) and a second frequency to affect bacteria or biofilm attachment to a foreign surface (e.g. metal or plastic). This could be done with pulsatile fluid suction and irrigation. This could also be done with subsonic, EMR, radiation, or electroshock therapy.

This technology could be a part of a multimodal treatment with any combination of ultrasound, vibratory, pulsed electromagnetic fields (PEMF), electroshock, pharmaceutical, phototherapy, or thermal treatments.

The systems and methods can also be used to dilate the cell membrane coatings. The vibratory frequency could optimize cell wall/membrane permeability to enhance local effect. This could also be used for autoimmune disease to enhance the effect. For example, if a joint has rheumatoid arthritis, one could use ultrasound and the thermal effect could decrease symptomatology and decrease fluid flow as well as enhance and localize the effect of the pharmaceutical agents from inflammatory diseases at specific locations, i.e. skin, joint, lungs, sclarea, derma, etc. This could also be done in combination with the treatments to enhance flow. For example, if one has pulmonary hypertension in later stages because of pulmonary fibrosis, ultrasound treatment could enhance airflow through the lungs as well as vascular permeability or vascular flow so one would decrease the pulmonary hypertension. Areas of pulmonary sclerosis could be used as an adjutant for pharmaceutical management to decrease the degree of pulmonary hypertension.

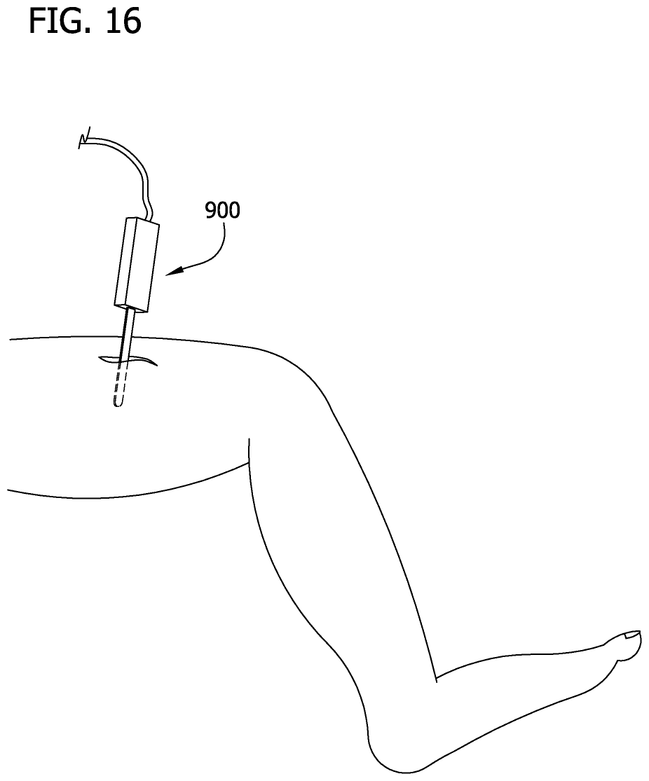

It should be noted that the systems and methods described herein are not limited to transdermal use. FIG. 16 is a perspective view of an alternative HFUS device 900 having the components of device 100 shown in FIG. 1. In the exemplary embodiment, the features of device 100 are integrated into a single handheld unit that is designed for an intracorporeal and/or percutaneous use to affect bacteria, biofilm, and/or infection within the body. In such an embodiment, at least a portion of applicator 104 is located and/or positioned in the body to provide HFUS. However, it should be noted that any portion of applicator can be inserted in any body portion to provide HFUS. The applicator 104 could be placed in body orifices, through an incision, or through cannula or expanding cannula.

In one embodiment, device 100 may include a light device, an endoscope, a fiber optic device, and/or other medical viewing devices to aid in viewing the treatment site inside the subject's body. In one example, the medical viewing device may be associated with applicator 104. In one particular example, applicator 104 may be provided on a distal end of a catheter, which includes the medical view device, for insertion into the subject's body. In other examples, the medical viewing device may be separate from applicator 104. Device 100 may include a specific modality for viewing of the treatment site using the viewing device. Ultrasound could also mark tissue to allow visualization of the cells or tissue during an endoscopic procedure. In addition, additional imaging may be used in conjunction with device 100, such as but not limited to MRI, CT, and PET scans of the treatment site.

In one embodiment, device 100 may include a fluid delivery device for delivering fluid to the treatment site. For example, the fluid delivery device may be configured for irrigating the treatment site and/or delivering pharmaceuticals or other substances to the treatment site. In one example, the medical viewing device may be associated with applicator 104 such that the applicator and the fluid delivery device can be used simultaneously or during the same treatment. In one particular example, applicator 104 may be provided on a distal end of a catheter, which includes the fluid delivery device, for insertion into the subject's body. For example, where the modality of device 100 causes delamination of biofilm from an implant or other surface, the fluid delivery device can be used to remove the delaminated biofilm from the body. In other examples, the fluid delivery device may be separate from applicator 104. Device 100 may include a specific modality for delivery of fluid to the treatment site using the fluid delivery device.

In one embodiment, device 900 is configured to be utilized in port sites within the body. For example, device 900 can be utilized with port sites including, but not limited to, catheter infusion sites, dialysis ports, and insulin ports, to treat and prevent infection. Such an embodiment can aid in ensuring the port or infusion site does not become infected or can be treated if it does get infected without actually removing the device that is inserted in the site. In some embodiments, output from device 900 is utilized after body piercing and/or tattooing to prevent or reduce the chance of infection. The device could be used for diagnostics or it could be used with a Smart Phone. The device 900 could be wireless with an endpiece that is operatively connected to a sensor that is implantable on the skin or in the body for sensing a body function or a fluid function, such as, chemistries, diabetes, blood sugar, oxygen tension, etc. Any implantable component could be partially biodegradable. Moreover, the device 900 or an associated end piece could be configured to use suction to adhere to the body to operatively position the sensors or hold the endpiece in position.

In one embodiment, device 100 may be configured to apply ultrasound topically or transdermally through small cannula with transducer 310 closer to the treatment site. In such an embodiment, there would be fluid inflow and fluid egress which would flush out the treatment area. During the flushing process, pharmaceuticals or other medicinal agent may be delivered locally or intravenously and the ultrasound would enhance the effect of the medicinal agent locally or in systemic pharmaceutical delivery. For example, stem cells may be delivered for chemotherapy. This could also be utilized for treatment or management of cancer. This could be used under image guidance. The ultrasound could be transmitted at the skin or through the skin to reach an implant or tissue. Characteristics of the ultrasound could be time varied based on the material property/thickness or biofilm materials could be separated. This could be done using two or more transducers at different locations.

As set forth above, applicator 104 may include imaging transducer 311. Ultrasound from imaging transducer 311 can be used purely diagnostically as the ultrasound may be able to have a different echogenic effect. For example, if there is biofilm attached to an implant, the ultrasound can detect the biofilm and device 100 would inform the surgeon the implant has an infection and should be treated in a different way. For example, an implant surface that is normal, without biofilm, would have one type of echogenicity, while an implant that has biofilm on it would have a different echogenicity because the biofilm would dampen the reflected signal received by imaging transducer 311. Thus, device 100 would inform the user that 1) the treatment site is infected and 2) biofilm treatment is required. The user then uses device 100 in the modality for treating biofilm so that biofilm can be removed from the implant, tissue or graft, without having to remove the implant. This may require one treatment or may require multiple treatments.

A specific area in which ultrasonic imaging and ultrasonic treatment of biofilm using device 100 is appropriate is total knee replacement. Typically, when there is an infection associated with a total knee replacement that lasts more than 2-4 weeks, there is an assumption that biofilm can grow on the implant and the entire implant needs to be removed. However, in one example using device 100, one can leave the implant in position, treat the implant with ultrasound to remove the biofilm and all infectious agents, and then flush/irrigate the treatment site with antibiotics or other treatment agents. This procedure could be done either endoscopically or arthroscopically without having to remove the implant. For example, in diabetics, device 100 could be placed on a CPM (continuous passive motion device), which moves the joint through flexion and extension during treatment. The ultrasound may be positioned superficially and may be placed at different positions around the joint. Treatment time may be for several minutes in one location and then the position may be changed 90 degrees relative to an axis of the knee. The knee may be treated for several minutes at the second location and then rotated another 90 degrees. This may be repeated at, for example, 3-4 positions around the knee so that the biofilm could be treated around the entire circumference of the knee. This could also be done during endoscopic or arthroscopic surgery where ultrasound would be administered percutaneously, which allows the applicator 104 to be positioned closer to the implant in a fluent environment. With the applicator positioned in the percutaneous port, the joint would be moved through a range of motion so the ultrasonic vibration would help multiple surfaces of the implant to remove biofilm wherever it is attached to the implant and not simply in one location. Ultrasound delivery at the site of a joint replacement may require a placement of one ultrasonic transducer at a single location, or one or more transducers could be placed at 3 or 4 positions around the joint (e.g., circumferentially around the knee) for selected periods of time (e.g., 3, 5, or 10 minutes). A joint implant would be treated with one or multiple treatments to remove the biofilm. The treatment site may also be imaged using device 100 to determine if the treatment is effective and the biofilm is being removed. This procedure could also be used for stents, cardiac valves, grafts, bone grafts, tissue grafts, cages, etc.

Moreover, if bacteria is also adherent or if there is bacteria in the fluid of the joint, the fluid changes and its viscosity becomes thicker, sludgy, more opaque rather than simple bleeding, hematoma, or fluid around the joint which is usually clear yellowish fluid. Device 100 may be configured for determining if the fluid is denser or thicker and more viscous, indicating there is an infection. Ultrasound can be used to pick up more infectious agents and these echogenicity patterns could be cataloged and stored. If there is a difference in echogenicity of a fluid area, this would also be an indicator for infection and then device 100 may be configured to initiate treatments. For example, device 100 may be configured to be capable of changing the ultrasonic frequency from a first frequency associated with a diagnostic modality to a second frequency associated with a treatment modality to begin treatment of infection. As set forth above, the ultrasonic treatment may be pulsed or constant. It could be varied through several different frequencies to enhance the removal of biofilm. Since the tissue may be in a three-dimensional location, treatment with ultrasound may be made at varying angles. In the sinuses, for example, one location may be treated, but it may require multiple treatments at multiple locations. Each of the separate sinuses may need to be treated, and ultrasound may be applied at each different sinus location. Two ultrasounds or multiple ultrasounds may be applied to one location simultaneously or in a stable, staggered fashion. There may be two separate locations to stabilize and/or enhance the treatment.

As an example, a jig or fixture may be used on the surface of the body or within the body to ensure the various angles are reproducibly performed for a period of time. The applicator 104 is stabilized against the surface for a period of time and then moved to the next position so the three-dimensional construct is fully treated for biofilm. The jig (or simply landmarks) may be used to position and hold applicator 104 for necessary intervals of time whether it is 1 minute, 5 minutes, or 10 minutes. Treatment may need to be applied hourly or 2-3 times a day for 5-7 days for complete treatment. Device 100 or just applicator 104 can be incorporated with an external brace, an external jig or a fixator. Transducer(s) 310 could be implanted internally and then external energy could be applied to the internal transducer that is implanted. This would act as an energy directing device to direct the ultrasonic field to the specific location.

As disclosed above, in addition to bacterial infection, device 100 may be used for infections caused by virus, fungus, and/or mycoplasma, for example. Device 100 may also be used for chemotherapy for rapidly multiplying cells like tumor cells. With the tumor cells' ability to adhere, one could selectively modulate the frequency. When tumor cells grow very rapidly, they can become resistant to certain chemotherapy agents. Ultrasound and/or combination therapy as described herein may make these cancer cells more sensitive, especially if ultrasound is applied at a specific location and a specific depth.

Ultrasound can be applied in two or more specific locations, e.g., wrapped around the body to apply ultrasound to multiple specific locations in the body. Ultrasound can also be applied internally or percutaneously via a probe so it is closer to the affected site. For example, multiple probes may be offset 90 degrees to each other or a predetermined distance from one another (e.g., 1 centimeter) depending on drive frequency and location so that the probes can have a specific or more focused effect. This could also be done with ultrasound and/or resistive heating, pH sensitivity, pH deposits with pH releases, etc. This can be performed with pulsed electron therapy, such as cold plasma. This could be done in combination with ultrasound.

In one embodiment, device 100 may be used to treat a subject with an acute cardiac event (e.g., heart attack). During a heart attack, the cardiac vessels go into spasm and the blood sludges through the area of the heart. Using device 100, ultrasound can be applied to the heart area to cause vascular dilatation. The blood vessel would dilate rather than contract and spasm allowing relaxation of the blood vessel and then it could enhance the blood flow through the coronary artery or veins to decrease the risk of damage during a cardiac event (e.g., heart attack). Ultrasound from device 100 can also be used to treat deep vein thrombosis or pulmonary emboli. If a blood clot goes to the heart and then to the lungs, this area could clot off. If the area is acutely treated, the vascular spasm would relax and may enhance breaking up with or without thrombolytic treatments like Coumadin, Streptokinase, Lovenox, fractionated heparins, etc. One could relax the vessels to decrease spasticity and/or dilate the vessels to decrease the damage from the acute vascular event. Ultrasound could be used for peripheral vascular treatments or acute cardiovascular events. It could also be used for strokes. If a clot is present in the cranium of the skull, an implantable device could be used to percutaneously introduce ultrasound to treat the local area. If ultrasound is deliverable to the target area without bone, metal, or other artifact in the way, then one could directly treat the area using topical ultrasound application. If there is bone in the way or metallic device in the way, one may use percutaneous ultrasound using device 100 which could be inserted close to the target area. The ultrasonic energy could also be applied to treat spasticity to 1) dilate the vessel, 2) enhance blood flow, and/or 3) potentially breakup a clot. Vascular ultrasound could also be used with pulsatile treatments, other chemotherapeutic agents, or anti-thrombolytic treatments.

Device 100 could also be used as previously discussed to potentially prevent blood clots by keeping vessels and flow moving. This could be used topically around the extremities, for example, to prevent DVT after surgery or even during a surgical procedure. The ultrasound could be applied for periods of time, either constantly or intermittently, every hour or two, to a specific area targeting veins to decrease risks of deep vein thrombosis by dilating and causing fluid flow. This could be done in addition to pulsatile stockings or other thrombolytic agents. The device 100 can also be used to break up clots, knots, and mucus. It could liquefy such materials and improve fluid flow. This could be done in conjunction with suction or pressure. This could also be used in a stent with balloon dilatation via wire. The device 100 could be used for vascular treatments in a transcutaneous or a percutaneous manner.

It is also considered that a properly configured waveform could selectively disrupt or change the rate of the DNA to RNA to protein sequence. This could be used for therapeutic purposes, but also as an adjunct to diagnosis by allowing the technician to selectively modify the growth rate of certain specimens.

Although the device 100 has been described above as outputting a HFUS waveform optimized to treat infection in a body, device 100 can be configured to output a LFUS waveform optimized to treat infection in a body.

The embodiments described herein may utilize executable instructions embodied in a non-transitory computer readable medium, including, without limitation, a storage device or a memory area of a computing device. Such instructions, when executed by one or more processors, cause the processor(s) to perform at least a portion of the methods described herein. As used herein, a “storage device” or “memory” is a tangible article, such as a hard drive, a solid state memory device, and/or an optical disk that is operable to store data.

Although specific features of various embodiments of the invention may be shown in some drawings and not in others, this is for convenience only. In accordance with the principles of the invention, any feature of a drawing may be referenced and/or claimed in combination with any feature of any other drawing.

This written description uses examples to disclose various embodiments, which include the best mode, to enable any person skilled in the art to practice those embodiments, including making and using any devices or systems and performing any incorporated methods.

1-26. (canceled)

27. An ultrasound treatment device configured to be used as a self-treatment device for a user, the ultrasound treatment device comprising:
a hand-held treatment applicator configured to be held by the user, wherein the treatment applicator has a distal end configured to acoustically couple with skin of the user as the applicator is being held by the user;
an ultrasound transducer disposed in the applicator, wherein the ultrasound transducer is coupled to the applicator to deliver the generated ultrasound energy through the distal end of the application and through the skin of the user into a treatment site of the user when the hand-held treatment applicator is being held by the user and the ultrasound transducer is sufficiently acoustically coupled with the skin the user via the hand-held treatment applicator;
a vibrator disposed in the applicator, the vibrator configured to generate vibratory energy, wherein the vibrator is coupled to the applicator to deliver the generated vibratory energy through the distal end of the applicator to the skin of the user to provide haptic vibrations to the user when the ultrasound transducer is acoustically coupled with the skin the user and is being held by the user;
a control circuit operatively connected to the ultrasound transducer and the vibrator, wherein the control circuit is configured to i) selectively supply electrical energy to the ultrasound transducer to generate the ultrasound energy having a frequency from about 1 MHz to about 5 MHz and ii) selectively supply electrical energy to the vibrator to generate the haptic vibrations,
wherein the control circuit is configured to, before generating the ultrasound energy having a frequency from about 1 MHz to about 5 MHz and the haptic vibrations, i) receive an impedance feedback signal indicative of acoustic coupling of the ultrasound transducer to the skin of the user and ii) determine, based on the received impedance feedback signal, if the ultrasound transducer is sufficiently acoustically coupled with the skin of the user for delivering the ultrasound energy to the treatment site,
wherein the control circuit is configured to automatically and simultaneously generate i) the ultrasound energy from the ultrasound transducer and ii) the haptic vibrations from the vibrator, if and only if the control circuit determines that the ultrasound transducer is sufficiently acoustically coupled with the skin of the user, whereby the ultrasound energy is delivered to the treatment site for ultrasound treatment and the haptic vibrations are delivered to the skin for haptic feedback only when the ultrasound transducer is sufficiently acoustically interfaced with the skin of the user,
wherein during generation of the ultrasound energy and the haptic vibrations, the control circuit is configured to i) continuously determine if the ultrasound transducer is acoustically coupled to the skin of the user based on the impedance feedback signal, and ii) automatically and simultaneously cease generation of the ultrasound energy from the ultrasound transducer and the haptic vibrations from the vibrator to thereby indicate to the user that ultrasound is not being delivered to the treatment site.

28. The ultrasound treatment device set forth in claim 27, wherein the treatment site is a sinus of the user and the skin is skin on the face of the user corresponding to location of the sinus.

29. The ultrasound treatment device set forth in claim 28, wherein the ultrasound energy provided to the sinus of the user is suitable to provide treatment to the sinus.

30. The ultrasound treatment device set forth in claim 27, wherein the treatment applicator includes a transmission component and a head portion, wherein the transmission component is formed of a different material than the head portion, wherein the transmission component defines the distal end of the treatment applicator.

31. The ultrasound treatment device set forth in claim 30, wherein the transmission component is formed from a plastic material.

32. The ultrasound treatment device set forth in claim 31, wherein the head portion is formed from a plastic material different from the plastic material of the transmission component.

33. The ultrasonic treatment device set forth in claim 27, further comprising a device housing separate from the treatment applicator, wherein the control circuit is housed within the device housing, wherein the device housing includes a user interface, wherein the user interface includes a graphical representation of at least a portion of a human body.

34. The ultrasonic treatment device set forth in claim 27, wherein the control circuit is further configured to automatically and simultaneously generate i) the ultrasound energy from the ultrasound transducer and ii) the haptic vibrations from the vibrator for a predetermined period of time, after which the control circuit is configured to simultaneously cease generation of the ultrasound energy and the haptic vibrations.

35. A method of self-treatment of a user using the ultrasound treatment device of claim 27, the method comprising:
holding, by the user, the hand-held treatment applicator of the ultrasound treatment device;
acoustically coupling, by the user, the distal end of the hand-held treatment applicator with skin on a face of the user corresponding to a location of a sinus to be treated with the hand-held treatment applicator;
determining, by the control circuit, the ultrasound transducer is sufficiently acoustically coupled with the skin of the user based on the impedance feedback signal received by the control circuit; and
instructing, by the user, the control circuit to automatically and simultaneously generate i) ultrasound energy from the ultrasound transducer having a frequency from about 1 MHz to about 5 MHz, and ii) haptic vibrations from the vibrator after the control circuit determines that the ultrasound transducer is sufficiently acoustically coupled with the skin of the user; and
generating, by the control circuit after said determining the ultrasound transducer is sufficiently coupled and after said instructing the control circuit, i) the ultrasound energy having the frequency from about 1 MHz to about 5 MHz and ii) the haptic vibrations.

36. An ultrasound treatment device configured to be used as a self-treatment device for a user, the device comprising:
a hand-held treatment applicator configured to be held by the user, wherein the treatment applicator has a distal end configured to acoustically couple with skin of the user as the applicator is being held by the user;
an ultrasound transducer disposed in the applicator, wherein the ultrasound transducer is coupled to the applicator to deliver the generated ultrasound energy through the distal end of the application and through the skin of the user into a treatment site of the user when the hand-held treatment applicator is being held by the user and the ultrasound transducer is sufficiently acoustically coupled with the skin the user via the hand-held treatment applicator;
a control circuit operatively connected to the ultrasound transducer, wherein the control circuit is configured to selectively supply electrical energy to the ultrasound transducer to generate the ultrasound energy having a frequency from about 1 MHz to about 5 MHz; and
a device housing separate from the treatment applicator, the device housing including a user interface to enable the user to communicate with the control circuit for operating the ultrasound treatment device.

37. The ultrasound treatment device set forth in claim 36, wherein the treatment site is a sinus of the user and the skin is skin on the face of the user corresponding to location of the sinus, wherein the ultrasound energy provided to the sinus of the user is suitable to provide treatment to the sinus.

38. The ultrasound treatment device set forth in claim 36, wherein the user interface includes a graphical representation of at least a portion of a human body.

39. The ultrasound treatment device set forth in claim 38, wherein the graphical representation comprises a human face.

40. The ultrasound treatment device set forth in claim 38, wherein the user interface is configured to enable the user to select a treatment area, from a plurality of different treatment areas, for treatment using the treatment device, wherein the selection of the treatment area is communicated to the control circuit and is visually indicated on the graphical representation.

41. The ultrasound treatment device set forth in claim 40, wherein the control circuit is configured to select and run a predetermined ultrasonic treatment session, from a plurality of different ultrasonic treatment sessions, based on the selected treatment area.

42. The ultrasound treatment device set forth in claim 41, wherein the plurality of different ultrasonic treatment sessions includes different output parameters of the ultrasound transducer.

43. The ultrasound treatment device set forth in claim 40, wherein the graphical representation comprises a human face, wherein the plurality of treatment areas include frontal sinus and maxillary sinus areas corresponding to the respective frontal and maxillary sinuses, wherein the control circuit is configured to select and run a frontal sinus ultrasonic treatment session when the user selects the frontal sinus area, wherein the control circuit is configured to select and run a maxillary sinus ultrasonic treatment session when the user selects the maxillary sinus area.

44. The ultrasound treatment device set forth in claim 36, further comprising an eccentric motor disposed in the applicator and configured to provide haptic feedback to the user, wherein the control circuit is in electrical communication with eccentric motor.

45. A method of self-treatment of a user using an ultrasound treatment device, the method comprising:
holding, by the user, a hand-held treatment applicator of the ultrasound treatment device, wherein an ultrasound generator and a vibrator are disposed in the hand-held treatment applicator;
acoustically coupling, by the user, the distal end of the hand-held treatment applicator with skin on a face of the user corresponding to a location of a sinus to be treated with the hand-held treatment applicator;
determining, by the control circuit, the ultrasound transducer is sufficiently acoustically coupled with the skin of the user based on a coupling signal received by the control circuit; and
instructing, by the user via a user interface, the control circuit to automatically and simultaneously generate i) ultrasound energy from the ultrasound transducer having a frequency from about 1 MHz to about 5 MHz, and ii) haptic vibrations from the vibrator, after the control circuit determines that the ultrasound transducer is sufficiently acoustically coupled with the skin of the user; and
generating simultaneously, after said determining the ultrasound transducer is sufficiently coupled and after said instructing the control circuit, i) the ultrasound energy having the frequency from about 1 MHz to about 5 MHz and ii) the haptic vibrations.

46. The method set forth in claim 45, further comprising:
determining continuously, by the control circuit during the said generating simultaneously the ultrasound energy and the haptic vibrations, if the ultrasound transducer is acoustically coupled to the skin of the user based on the impedance feedback signal.
